(19) United States
(12) Patent Application Publication
(10) Pub. No.: US 2025/0059143 A1
(43) Pub. Date: Feb. 20, 2025

(54) CAMPTOTHECIN DERIVATIVE INTERMEDIATE, AND PREPARATION METHOD THEREFOR AND USE THEREOF

(71) Applicant: SHANGHAI BEST-LINK BIOSCIENCE, LLC, Shanghai (CN)

(72) Inventors: Fuyao ZHANG, Shanghai (CN); Xianjie CHEN, Shanghai (CN)

(21) Appl. No.: 18/701,611

(22) PCT Filed: Oct. 26, 2022
(86) PCT No.: PCT/CN2022/127677

§ 371 Date: Apr. 15, 2024

(30) Foreign Application Priority Data
Oct. 26, 2021 (CN) 202111248373.7

(51) Int. Cl. C07D 263/06; B01J 31/22; B01J 31/24; C07C 231/10; C07C 231/24; C07C 237/20; C07C 269/08; C07C 271/22; C07C 271/24; C07D 263/52; C07D 491/22; C07D 498/10; C07F 7/12; C07F 7/14

(52) U.S. Cl. CPC C07D 263/06; B01J 31/2295; B01J 31/2409; C07C 231/10; C07C 231/24; C07C 237/20; C07C 269/08; C07C 271/22; C07C 271/24; C07D 263/52; C07D 491/22; C07D 498/10; C07F 7/12; C07F 7/14; B01J 2231/4211; B01J 2531/004; B01J 2531/0205; B01J 2531/824; B01J 2531/842; C07C 2601/16; C07C 2602/10

(57) ABSTRACT

Disclosed in the present invention are a camptothecin derivative intermediate, and a preparation method therefor and the use thereof. The structural formula of the camptothecin derivative intermediate of the present invention is as shown in formula (I), and the definition of each substituent is as described in the description and claims. The intermediate (I) of the present invention can be used for preparing an intermediate (III), and can be further used for preparing exatecan and a derivative thereof. The preparation method of the present invention has the advantages of cheap and easily available raw materials, a novel method and simple route, mild conditions, a high yield, few by-products, suitability for scale-up synthesis and industrial production, etc.

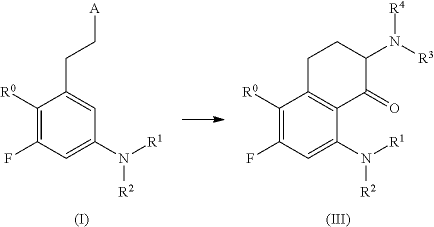

This application claims priority to Chinese Application No. 2021112483737, filed on Oct. 26, 2021, the entire disclosure of which is incorporated herein by reference.

### TECHNICAL FIELD

The present invention relates to the technical field of organic synthesis, specifically to a camptothecin derivative intermediate, its preparation method and application thereof, especially the preparation of exatecan.

### BACKGROUND

DS-8201a (trastuzumab deruxtecan) is a second-generation antibody drug conjugate (ADC) targeting HER2, which is composed of trastuzumab, linker and exatecan derivative (Dxd). It has been approved by the FDA for accelerated approval, fast-track designation and breakthrough therapy designation, and was approved by the FDA in 2019 to treat breast cancer, gastric or gastroesophageal adenocarcinoma. Compared with the first-generation ADC, DS-8201a has stronger anti-tumor potential, higher drug-antibody ratio DAR (7˜8), carries high drug efficacy and stronger membrane penetration of Dxd. After being released by lysosomes, it can penetrate into neighboring cells and kill non-targeted tumor cells, making it have a bystander effect, so it is also effective against breast cancer with low expression of HER2.

The structural formulas of Exatecan and Dxd are as follows:

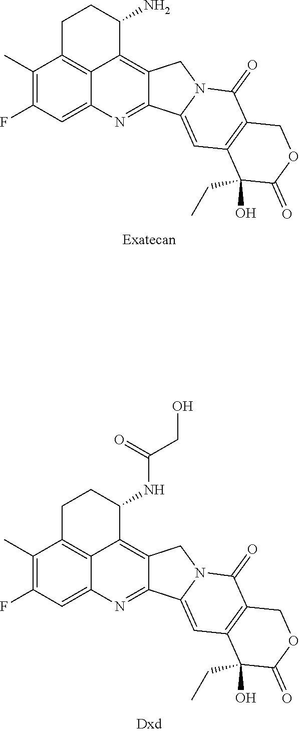 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3]

Exatecan is a new camptothecin derivative, which has a unique six-ring structure. In particular, the introduction of an amino group into the ring makes the compound have better water solubility, and the substituent of benzene ring can further delay the ring opening of lactone in vivo, which makes it highly effective.

The preparation of exatecan was disclosed in patents EP0495432B1 and EP2910573A1. It was obtained by ring-closing reaction and derivatization of intermediates 6 and 7 (see formula below).

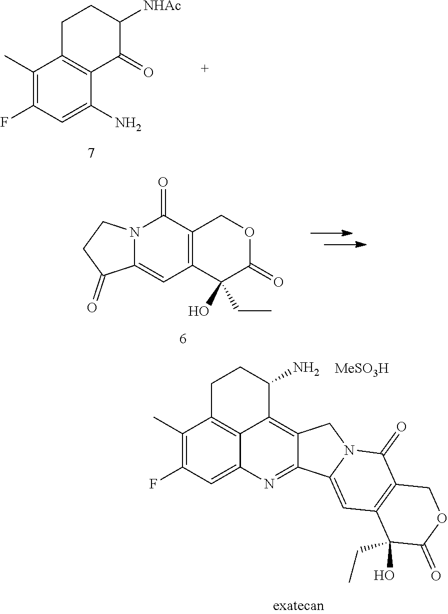

The preparation of key intermediate 7 was reported in patents EP0495432B1, WO1996026181A1, CN111470998 and TW201920078A. The methods were as follows: intermediate 8 was oxidized to oxime intermediate 9 by nitrous acid, then reduced to intermediate 10, and finally deprotected to obtain compound 7.

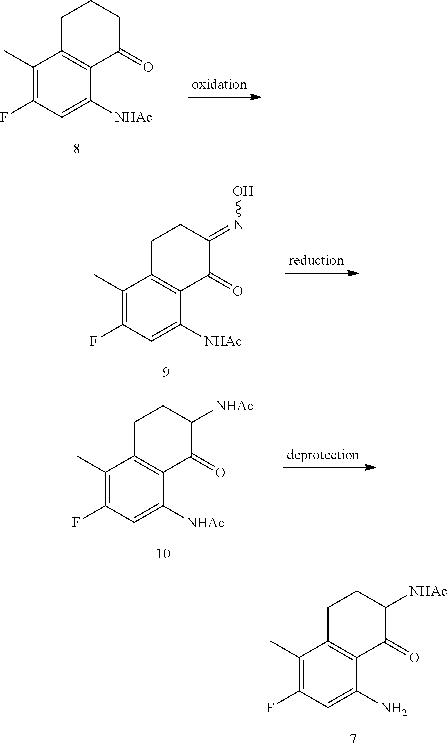

The synthetic method disclosed in the above patent has a long route, and the obtained intermediate 7 is racemic. However, the preparation method in this invention is to obtain racemic or chiral intermediate 10 with different protective groups by one-step ring closing reaction, and then obtain chiral or racemic key intermediate 7 by deprotection. The preparation method of the invention has the advantages of simple synthetic route, easily available raw materials, mild conditions, and simple post-treatment, and is suitable for amplification and industrial production.

### CONTENT OF THE PRESENT INVENTION

The purpose of the invention is to provide a new camptothecin derivative intermediate, its preparation method and application.

In one aspect, the present invention relates to a compound represented by formula (I), its stereoisomer or its pharmaceutically acceptable salt:

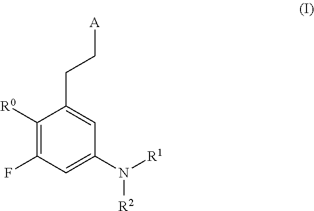

wherein,

A is selected from

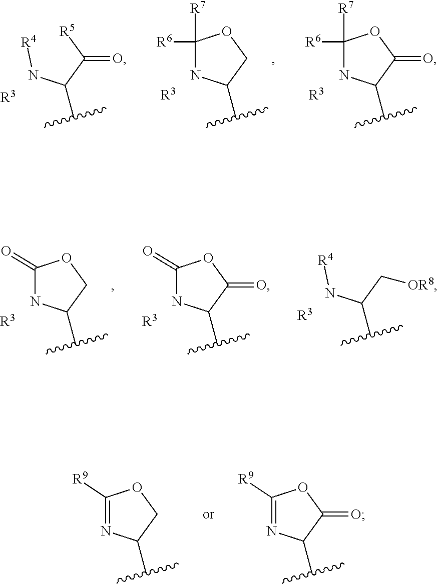

the chiral configuration in the A structure can be the R configuration, the S configuration, or a mixture of the R configuration and the S configuration;

R⁰ is selected from hydroxyl, alkoxy, sulfhydryl, alkylthio, halogen, nitro, substituted or unsubstituted amino, C1-C6 alkyl, C1-C6 cycloalkyl, C1-C6 haloalkyl, C1-C6 deuterium Alkyl, C2-C6 alkenyl or C2-C6 alkynyl;

R¹ and R² are each independently selected from hydrogen or amino protecting groups;

R³ and R⁴ are each independently selected from hydrogen or amino protecting groups;

R⁵ is selected from hydrogen, hydroxyl, alkoxy, alkylthio, halogen or substituted or unsubstituted amino;

R⁶ and R⁷ are each independently selected from hydrogen, alkyl, haloalkyl or deuterated alkyl;

alternatively, R⁶ and R⁷ together with the carbon atom linked thereto form a 3-7 membered cycloalkyl or heterocycloalkyl group;

R⁸ is selected from hydrogen or hydroxyl protecting group;

R⁹ is selected from hydrogen, alkoxy, alkylthio, C1-C6 alkyl, C1-C6 haloalkyl, aryl or heteroaryl.

In a preferred embodiment of the camptothecin derivative intermediate represented by formula (I) of the present invention, R⁰ is a methyl group.

The invention also provides a preparation method of the compound shown in formula (I), which is characterized by comprising the following steps: firstly, adding compound 2 with borane reagent to form a boron intermediate, and then carrying out a coupling reaction with compound 1 with metal catalyst and base, and the reaction formula is as follows:

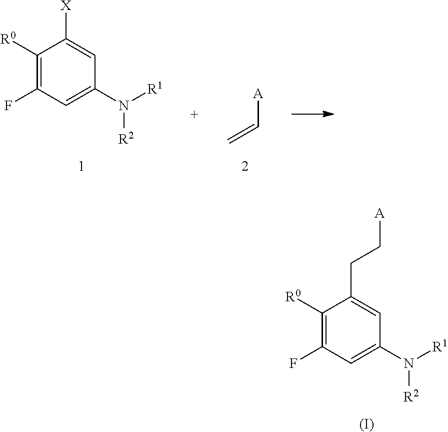

wherein, X is selected from Cl, Br or I;

R⁰, R¹, R² and A are each defined as defined in the general formula (I) of the present invention.

The invention also provides a preparation method of the compound shown in formula (I), which is characterized by comprising the following steps: firstly, compound 3 and zinc form a zinc intermediate with catalyst, and then is coupled with compound 1, and the reaction formula is as follows:

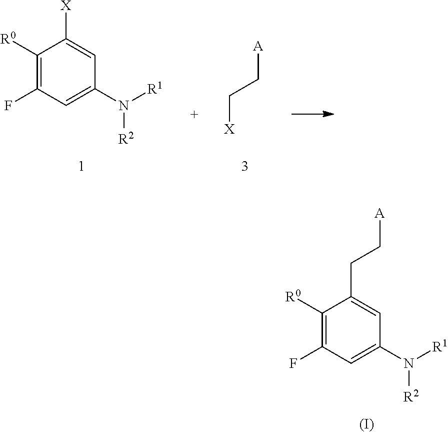

wherein X is selected from Cl, Br or I;

R⁰, R¹, R² and A are each defined as defined in the general formula (I) of the present invention.

The present invention also provides a preparing method of the compound shown in formula (I), which is characterized in that it includes the following steps: compound 3 first reacts with magnesium to form a magnesium intermediate or is exchanged with a Grignard reagent to form a magnesium intermediate, and then is coupled with compound 1. The reaction formula is as follows:

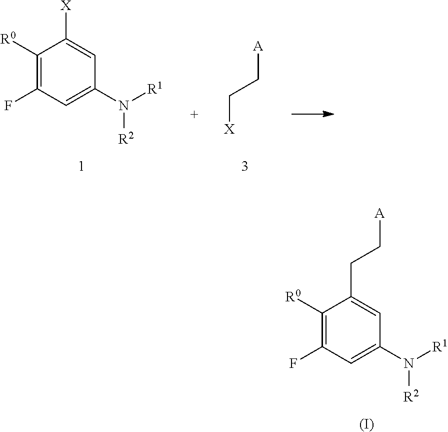

wherein, X is selected from Cl, Br or I;

R⁰, R¹, R² and A are each defined as in the general formula (I) of the present invention.

The invention also provides a preparation method of the compound shown in formula (I), which is characterized by comprising the following steps: firstly, compound 3 reacts with magnesium to form a magnesium intermediate, or exchanges with a Grignard reagent to form a magnesium intermediate, then exchanges with a zinc reagent to form a zinc intermediate, and then couples with compound 1, and the reaction formula is as follows:

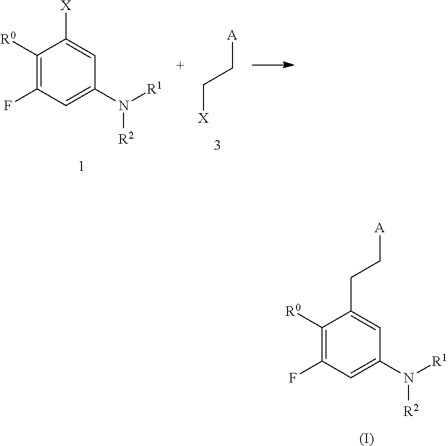

wherein, X is selected from Cl, Br or I;

R⁰, R¹, R² and A are each defined as in the general formula (I) of the present invention.

In a preferred embodiment of the preparation method of the camptothecin derivative intermediate represented by formula (I) of the present invention, R⁰ is a methyl group.

The invention also provides a method for preparing a compound of formula (II), a stereoisomer thereof or a pharmaceutically acceptable salt thereof from the compound of formula (I), which is characterized by comprising the following steps: the compound represented by formula (I) is converted into a compound of formula (II), and the reaction formula is as follows:

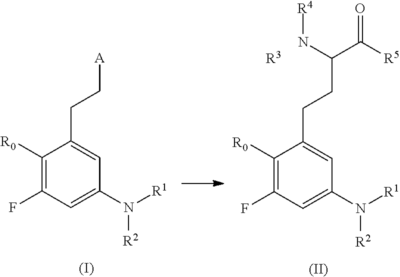

wherein R⁵ is selected from hydroxyl or halogen;

R⁰, R¹, R², R³, R⁴ and A are each defined as defined in the general formula (I) of the present invention.

In a preferred embodiment of the preparation method of the camptothecin derivative intermediate represented by formula (II) of the present invention, R⁰ is a methyl group.

The invention also provides a method for preparing the compound of formula (III), its stereoisomer or a pharmaceutically acceptable salt thereof from the compound of formula (II), characterized in that the compound of formula (II) is intramolecular cyclized with catalyst to generate a compound of formula (III), and the reaction formula is as follows:

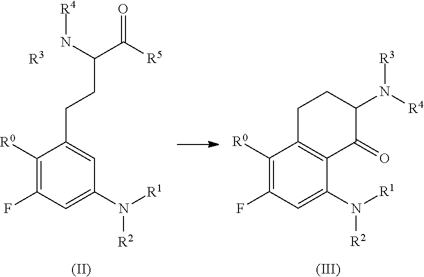

wherein R⁵ is selected from hydroxyl or halogen;

R⁰, R¹, R², R³ and R⁴ are each defined as defined in the general formula (I) of the present invention;

the chiral configuration of the compound (III) can be R configuration, S configuration or a mixture of R configuration and S configuration.

In a preferred embodiment of the preparation method of the camptothecin derivative intermediate represented by formula (III) of the present invention, R⁰ is a methyl group.

The present invention also provides a method for preparing the compound of formula (IV), its stereoisomer or its pharmaceutically acceptable salt from the compound represented by formula (III), which is characterized by comprising the following steps:
a. selectively deprotecting the compound shown in formula (III) to obtain compound 4;
b. compound 4 and compound 5 are cyclized with catalyst to generate compound 6;
c. removing the protecting group from the compound 6 to obtain the compound of formula (IV), its stereoisomer or its pharmaceutically acceptable salt.

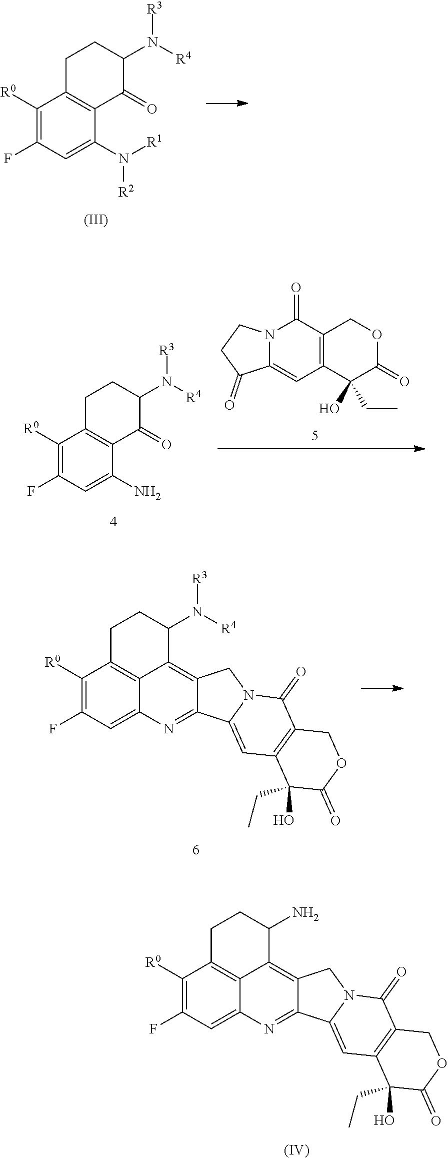

wherein,

R⁰, R¹, R², R³ and R⁴ are each defined as defined in the general formula (I) of the present invention.

In a preferred embodiment of the preparation method of the camptothecin derivative intermediate represented by formula (IV) of the present invention, R⁰ is a methyl group.

In a preferred embodiment of the preparation method of the camptothecin derivative intermediate represented by formula (IV) of the present invention, the chiral configuration of compound (III) is the R configuration.

In a preferred embodiment of the preparation method of the camptothecin derivative intermediate represented by formula (IV) of the present invention, the chiral configuration of compound (III) is the S configuration.

In a preferred embodiment of the preparation method of the camptothecin derivative intermediate represented by formula (IV) of the present invention, the chiral configuration of compound 4 is the R configuration.

In a preferred embodiment of the preparation method of the camptothecin derivative intermediate represented by formula (IV) of the present invention, the chiral configuration of compound 4 is the S configuration.

In a preferred embodiment of the present invention, the compound of formula (I) is selected from the following structures without limitation:

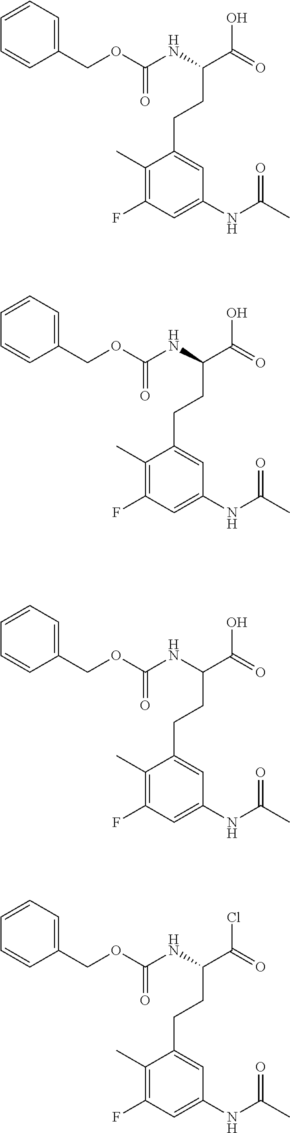 [SMILES: CC(=O)Nc1cc(F)c(C)c(CC[C@H](NC(=O)OCc2ccccc2)C(=O)O)c1 ; CC(=O)Nc1cc(F)c(C)c(CC[C@@H](NC(=O)OCc2ccccc2)C(=O)O)c1 ; CC(=O)Nc1cc(F)c(C)c(CCC(NC(=O)OCc2ccccc2)C(=O)O)c1 ; CC(=O)Nc1cc(F)c(C)c(CC[C@H](NC(=O)OCc2ccccc2)C(=O)Cl)c1]

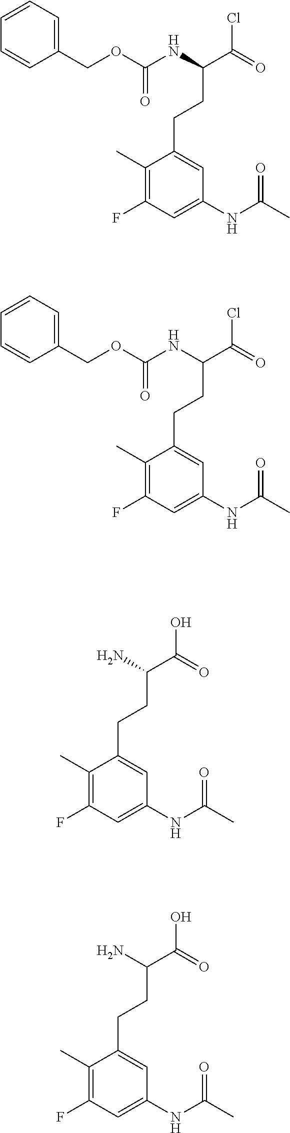

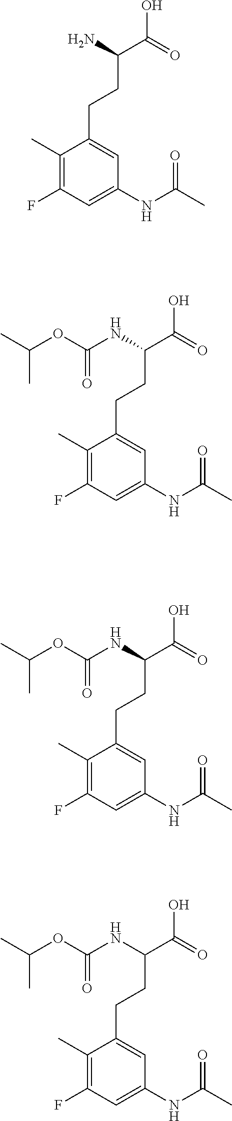

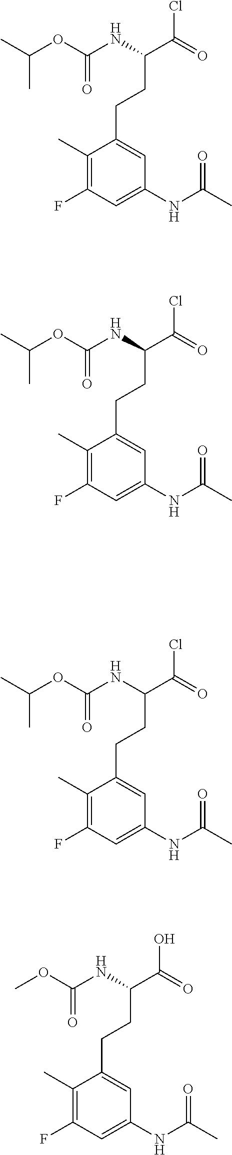

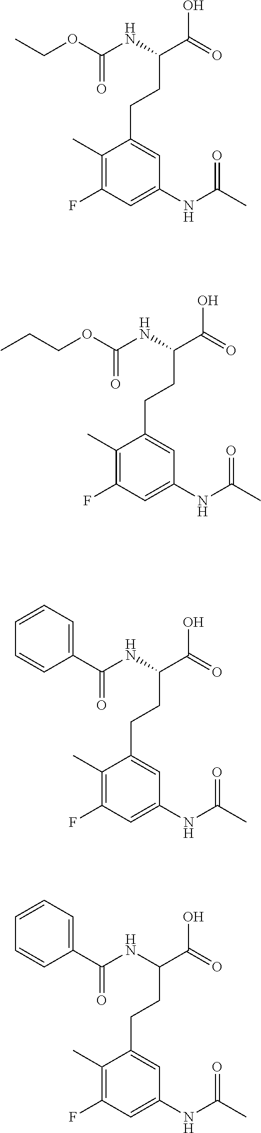

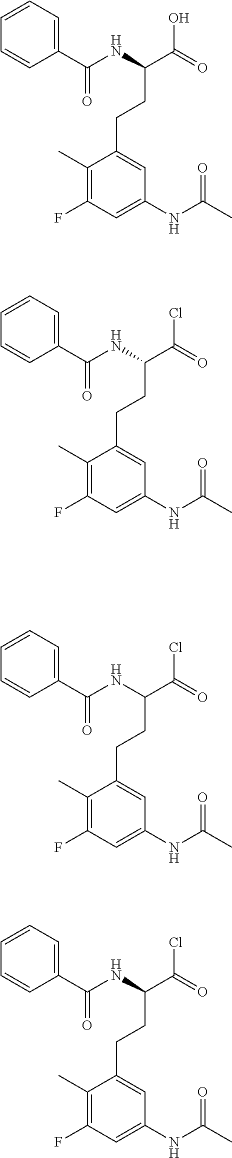 [SMILES: CC(=O)Nc1cc(F)c(C)c(CC[C@@H](NC(=O)c2ccccc2)C(=O)O)c1 ; CC(=O)Nc1cc(F)c(C)c(CC[C@H](NC(=O)c2ccccc2)C(=O)Cl)c1 ; CC(=O)Nc1cc(F)c(C)c(CCC(NC(=O)c2ccccc2)C(=O)Cl)c1 ; CC(=O)Nc1cc(F)c(C)c(CC[C@@H](NC(=O)c2ccccc2)C(=O)Cl)c1]

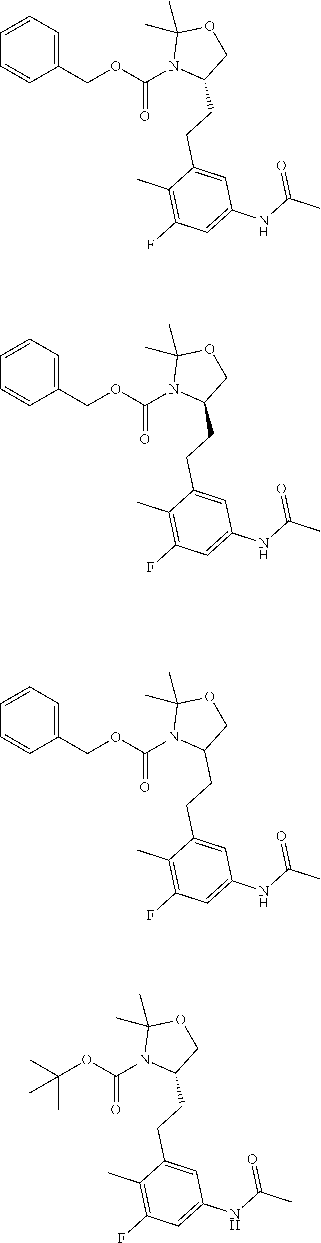

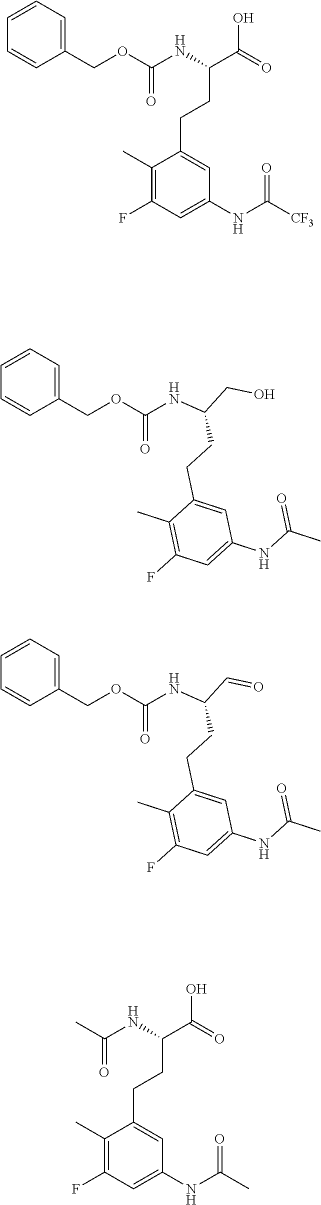

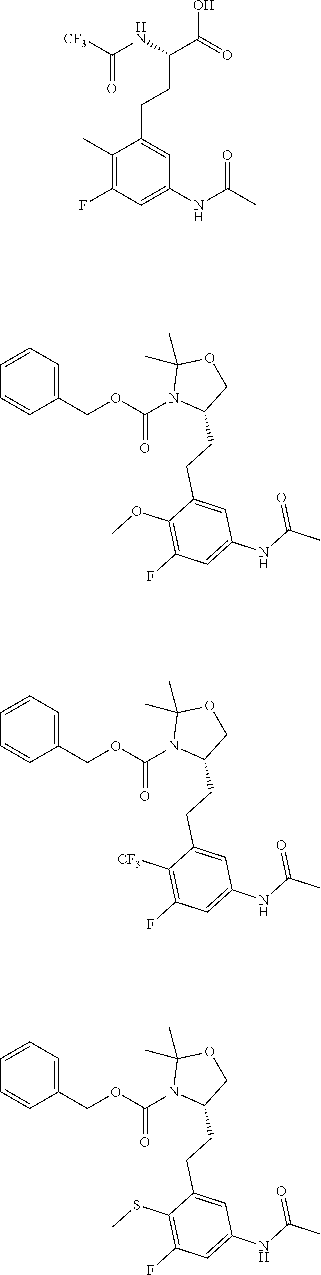 [SMILES: CC(=O)Nc1cc(F)c(C)c(CC[C@H](NC(=O)C(F)(F)F)C(=O)O)c1 ; COc1c(F)cc(NC(C)=O)cc1CC[C@H]1COC(C)(C)N1C(=O)OCc1ccccc1 ; CC(=O)Nc1cc(F)c(C(F)(F)F)c(CC[C@H]2COC(C)(C)N2C(=O)OCc2ccccc2)c1 ; CSc1c(F)cc(NC(C)=O)cc1CC[C@H]1COC(C)(C)N1C(=O)OCc1ccccc1]

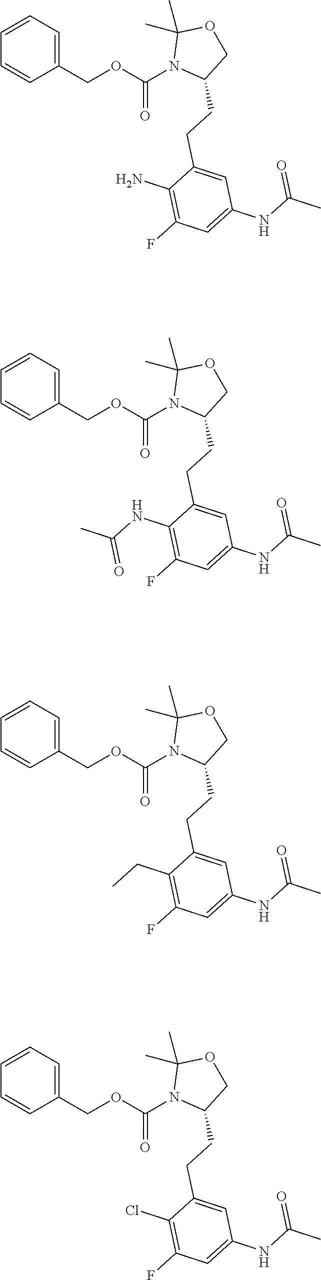 [SMILES: CC(=O)Nc1cc(F)c(N)c(CC[C@H]2COC(C)(C)N2C(=O)OCc2ccccc2)c1 ; CC(=O)Nc1cc(F)c(NC(C)=O)c(CC[C@H]2COC(C)(C)N2C(=O)OCc2ccccc2)c1 ; CCc1c(F)cc(NC(C)=O)cc1CC[C@H]1COC(C)(C)N1C(=O)OCc1ccccc1 ; CC(=O)Nc1cc(F)c(Cl)c(CC[C@H]2COC(C)(C)N2C(=O)OCc2ccccc2)c1]

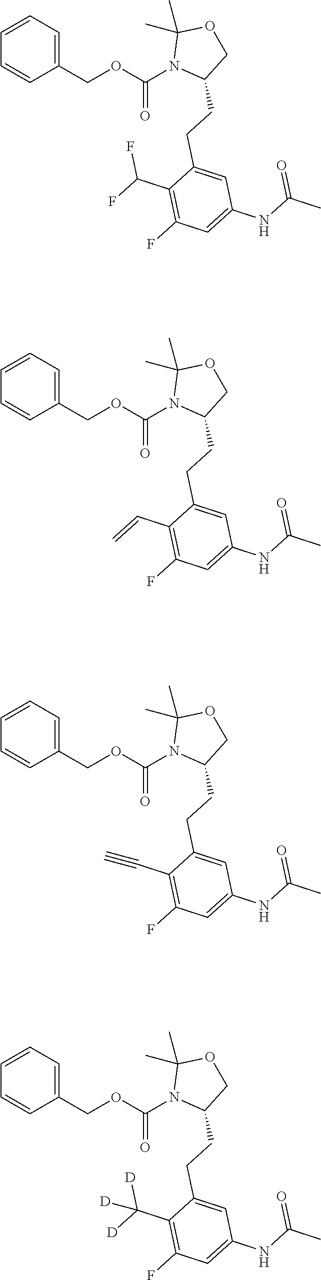 [SMILES: CC(=O)Nc1cc(F)c(C(F)F)c(CC[C@H]2COC(C)(C)N2C(=O)OCc2ccccc2)c1 ; C=Cc1c(F)cc(NC(C)=O)cc1CC[C@H]1COC(C)(C)N1C(=O)OCc1ccccc1 ; C#Cc1c(F)cc(NC(C)=O)cc1CC[C@H]1COC(C)(C)N1C(=O)OCc1ccccc1 ; [2H]C([2H])([2H])c1c(F)cc(NC(C)=O)cc1CC[C@H]1COC(C)(C)N1C(=O)OCc1ccccc1]

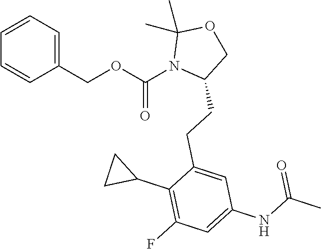 [SMILES: CC(=O)Nc1cc(F)c(C2CC2)c(CC[C@H]2COC(C)(C)N2C(=O)OCc2ccccc2)c1]

In a preferred embodiment of the present invention, the compound of formula (III) is selected from the following structures without limitation:

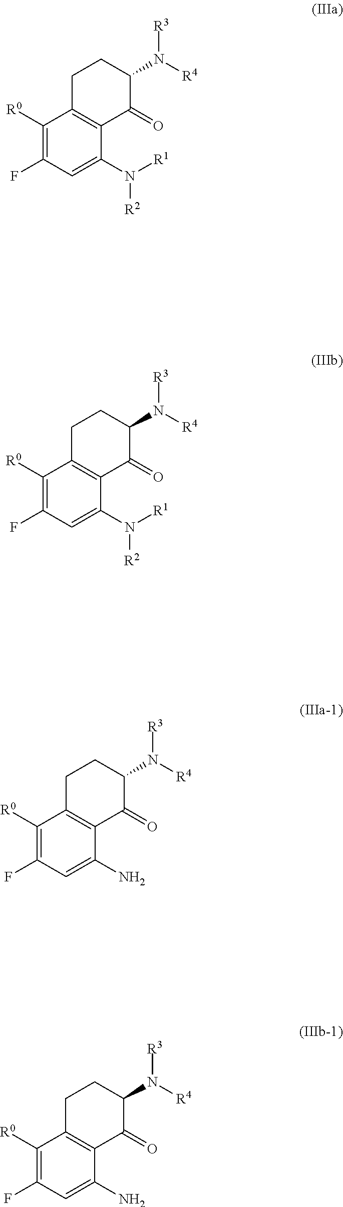

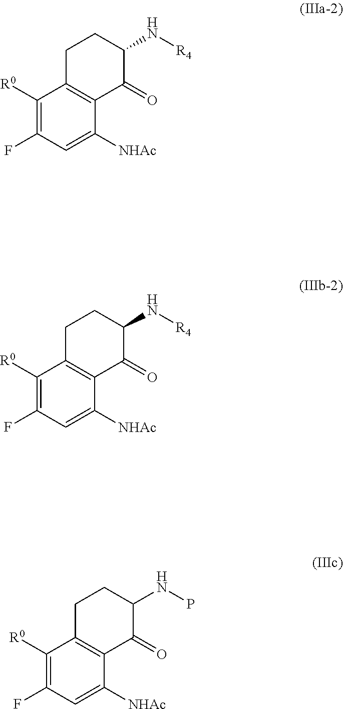

wherein, P is selected from amino protecting groups other than acetyl protecting groups;

R⁰, R¹, R², R³ and R⁴ are each defined as defined in the general formula (I) of the present invention.

In a preferred embodiment of the present invention, the borane reagent in the preparation method of the compound represented by formula (I) is 9-borabicyclo[3.3.1] nonane (9-BBN), dicyclohexylborane (Cy₂BH), catecholborane (CatBH), pinacol borane (PinBH) and disiamylborane (Sia₂BH), borane tetrahydrofuran complex, borane dimethyl sulfide complex or diisopinenylborane (Ipc₂BH); preferably, the borane reagent is 9-BBN.

In a preferred embodiment of the present invention, the coupling reaction in the method for preparing the compound represented by formula (I) is a Suzuki reaction, and the metal catalyst is selected from Pd(PPh₃)₄, PdCl₂(dppf), PdCl₂(dppf)CH₂Cl₂, PdCl₂(PPh3)₂, Pd₂(dba)₃/XPhos, Pd₂(dba)₃/sPhos, Pd₂(dba)₃/XantPhos, Pd(OAc)₂/PCy₃, PdCl₂(dppe) or PdCl₂(dppp), preferably Pd(PPh₃)₄.

The base is selected from one or more of potassium phosphate, sodium carbonate, cesium carbonate, potassium carbonate, sodium bicarbonate, potassium fluoride, cesium fluoride, sodium hydroxide, potassium hydroxide, sodium tert-butoxide, potassium tert-butoxide, potassium acetate, triethylamine, diisopropylethylamine and DBU, preferably potassium phosphate; the solvent is selected from one or more of tetrahydrofuran, methyltetrahydrofuran, diethyl ether, toluene, DMF, DMA, DME, NMP, DMSO, dioxane, 1,2-dichloroethane, tert-butanol, water and acetonitrile, preferably tetrahydrofuran, water and DMF.

In a preferred embodiment of the present invention, the catalyst used to form the zinc intermediate in the preparation method of the compound represented by formula (I) is selected from one or more of 1,2-dibromoethane, TMSCl, iodine, lithium chloride or lithium bromide, preferably 1,2-dibromoethane and TMSCl; the coupling reaction is Negishi coupling reaction, and the metal catalyst is selected from Pd(PPh₃)₄, PdCl₂(dppf), PdCl₂(dppf)CH₂Cl₂, PdCl₂(PPh₃)₂, Pd₂(dba)₃/XPhos, Pd₂(dba)₃/SPhos, Pd₂(dba)₃/XantPhos, Pd(OAc)₂PCy₃, Pd₂(dba)₃/P(o-tol)₃, Pd₂(dba)₃/P(o-tol)₃, Pd₂(dba)₃/P(2-furyl)₃, Pd₂(dba)₃/SPhos, Pd₂(dba)₃/RuPhos, PEPPSI-IPr, PEPPSI-IPent, NiCl₂(PPh₃)₂, preferably PEPPSI-IPr; the solvent is selected from one or more of tetrahydrofuran, methyltetrahydrofuran, toluene, DMF, DMA, DME, NMP, DMSO, dixoane, 1,2-dichloroethane, tert-butanol, water, and acetonitrile, preferably DMF.

In a preferred embodiment of the present invention, the Grignard reagent used to form the magnesium intermediate in the preparation method of the compound represented by formula (I) is selected from isopropyl magnesium chloride, isopropyl magnesium bromide, isopropyl magnesium bromide, and isopropyl magnesium chloride lithium chloride complex, ethyl magnesium bromide, ethyl magnesium chloride, phenyl magnesium chloride, phenyl magnesium bromide; the coupling reaction is Kumada coupling reaction, and the metal catalyst is selected from Pd(PPh₃)₄, NiCl₂(dppp), NiCl₂(dppe), NiCl₂(PPh₃)₂, ferric acetylacetonate, nickel(II) acetylacetonate, FeCl₃, CoCl₂, Pd(PPh₃)₄, PdCl₂(dppf), PdCl₂(dppf)CH₂Cl₂, Pd(OAc)₂/PCy₃, Pd₂(dba)₃/SPhos; the solvent is selected from tetrahydrofuran, methyltetrahydrofuran, diethyl ether, toluene, DMF, DMA, DME, NMP, DMSO, dioxane, 1,2-dichloroethane, tert-butanol, water, and acetonitrile, preferably tetrahydrofuran.

In a preferred embodiment of the present invention, the zinc reagent described in the method for preparing the compound represented by formula (I) is selected from zinc chloride, zinc chloride-TMEDA, zinc bromide, zinc iodide, zinc methoxide; the coupling reaction is Negishi coupling reaction, and the metal catalyst is selected from Pd(PPh₃)₄, PdCl₂(dppf), PdCl₂(dppf)CH₂Cl₂, PdCl₂(PPh₃)₂, Pd₂(dba)₃/XPhos, Pd₂(dba)₃/SPhos, Pd₂(dba)₃/XantPhos, Pd(OAc)₂/PCy₃, Pd₂(dba)₃/P(o-tol)₃, Pd₂(dba)₃/P(o-tol)₃, Pd₂(dba)₃/P(2-furyl)₃, Pd₂(dba)₃/SPhos, Pd₂(dba)₃/RuPhos, PEPPSI-IPr, PEPPSI-IPent, NiCl₂(PPh₃)₂, preferably PEPPSI-IPr.

In a preferred embodiment of the present invention, the catalyst used in the ring closing reaction in the preparation method of the compound represented by formula (III) is selected from one or more of anhydrous aluminum trichloride, tin tetrachloride, and titanium tetrachloride, ferric chloride, boron trifluoride ether, trifluoroacetic anhydride, concentrated sulfuric acid, and hexafluoroisopropanol, preferably anhydrous aluminum trichloride.

### DETAILED DESCRIPTION OF THE INVENTION

Unless otherwise stated to the contrary, the terms used in the specification and claims have the following definitions.

The term “alkyl” refers to a saturated aliphatic hydrocarbon group, including linear or branched groups of 1 to 20 carbon atoms, preferably 1 to 10 carbon atoms, more preferably 1 to 8 carbon atoms, and non-limiting examples include, but are not limited to, methyl, ethyl, n-propyl, n-butyl, isobutyl, ter-butyl, sec-butyl, n-pentyl, 2-methylbutyl, 3-methylbutyl, 1,1-dimethylpropyl, 1,2-dimethylpropyl, 2,2-dimethylpropyl, 1-ethylpropyl, n-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1-ethyl-2-methylpropyl, n-heptyl, 2-methylhexyl, 3-methylhexyl, 4-methylhexyl, 5-methylhexyl, 2,2-dimethylpentyl, 2,3-dimethylpentyl, 2,3-dimethylpentyl, 2,4-dimethylpentyl, 3,3-dimethylpentyl, 3,4-dimethylpentyl, 2-ethylpentyl, 3-ethylpentyl, octyl, nonyl, decyl, undecyl, dodecyl, and various isomers thereof, and the like. The alkyl can be substituted or unsubstituted and can be substituted at any available junction, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkyloxy, heterocycloalkyloxy, cycloalkylmercapto, heterocycloalkylmercapto, oxo, amino, haloalkyl, hydroxyalkyl, carboxyl or carboxylate group, etc. When “alkyl” and its prefix are used herein, both linear and branched saturated carbon bonds are included.

The term “cycloalkyl” refers to a saturated or partially unsaturated monocyclic or polycyclic group comprising from 3 to 20 carbon atoms, preferably 3 to 12 carbon atoms, more preferably 3 to 10 carbon atoms, and most preferably 3 to 6 carbon atoms. Non-limiting examples of monocyclic cycloalkyl include, but are not limited to, cyclopropyl, cyclobutyl, cyclopentyl, cyclopentenyl, cyclohexyl, cyclohexenyl, cyclohexadiene, cycloheptyl, cyclooctyl, and the like. Non-limiting examples of polycyclic cycloalkyl include, but are not limited to, spirocycloalkyl, fused cycloalkyl and bridged cycloalkyl. Cycloalkyl can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy, cycloalkoxy, cycloalkoxy, cycloalkoxy, cycloalkylmercapto, heterocycloalkylmercapto, oxo, amino, haloalkyl, hydroxyalkyl, carboxyl or carboxylate group.

The term “haloalkyl” refers to an alkyl can be substituted by one or more than one the same or different halogen atoms, wherein the definition of the alkyl is as defined herein.

The term “deuterated alkane” means that an alkyl group may be substituted by one or more deuterium atoms, wherein alkyl group is as defined herein.

The term “alkenyl” refers to an alkyl as defined in the present invention consisting of at least two carbon atoms and at least one carbon-carbon double bond, preferably C₂-C₁₀ alkenyl, more preferably C₂-C₆ alkenyl, most preferably C₂-C₄ alkenyl, such as vinyl, propenyl, 1-propenyl, and the like. The alkenyl group can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy, cycloalkoxy, cycloalkoxy, cycloalkoxy, cycloalkylmercapto, heterocycloalkylmercapto, oxo, amino, haloalkyl, hydroxyalkyl, carboxyl or carboxylate group.

The term “alkynyl” refers to an alkyl as defined in the present invention consisting of at least two carbon atoms and at least one carbon-carbon triple bond, preferably C₂-C₁₀ alkynyl, more preferably C₂-C₆ alkynyl, most preferably C₂-C₄ alkynyl, such as ethynyl, 1-propynyl, 2-propynyl, and the like. The alkynyl group can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy, cycloalkoxy, cycloalkoxy, cycloalkoxy, cycloalkylmercapto, heterocycloalkylmercapto, oxo, amino, haloalkyl, hydroxyalkyl, carboxyl or carboxylate group.

The term “heterocycloalkyl” refers to a saturated or partially unsaturated monocyclic or polycyclic hydrocarbon group comprising from 3 to 20 ring atoms, wherein one or more than one ring atoms are selected from heteroatoms of N, O, S(O)ₘ, P(O)ₘ (wherein m is an integer from 0 to 2), but excluding ring moiety of —O—O, —O—S— or —S—S— and the remaining ring atoms are carbon. Preferably 3 to 12 ring atoms containing 1 to 4 heteroatoms, and non-limiting examples of monocyclic heterocycloalkyl include pyrrolyl, piperidinyl, piperazinyl, morpholinyl, tetrahydrofuranyl, pyranyl, and the like. Polycyclic heterocycloalkyl include spiro heterocycloalkyl, fused heterocycloalkyl and bridged heterocycloalkyl. Heterocycloalkyl can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkyloxy, heterocycloalkyloxy, cycloalkylmercapto, heterocycloalkylmercapto, oxo, amino, haloalkyl, hydroxyalkyl, carboxyl or carboxylate group.

The term “alkoxy” refers to —O-(alkyl) and —O-(cycloalkyl), wherein the definitions of the alkyl and the cycloalkyl are as described in the description. Non-limiting examples include, but are not limited to, methoxy, ethoxy, propoxy, butoxy, cyclopropoxy, cyclobutoxy, cyclopentyloxy, cyclohexyloxy, and the like. The alkoxy group can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkyloxy, heterocycloalkyloxy, heterocycloalkyloxy, cycloalkylmercapto, heterocycloalkylmercapto, oxo, amino, haloalkyl, hydroxyalkyl, carboxyl or carboxylate group.

The term “alkylmercapto” refers to —S-(alkyl) and —S-(cycloalkyl), wherein the definitions of the alkyl and the cycloalkyl are as described in the description. Non-limiting examples include, but are not limited to, methylmercapto, ethylmercapto, propylmercapto, butylmercapto, cyclopropylmercapto, cyclobutylmercapto, cyclopentylmercapto, cyclohexylmercapto, and the like. Alkylmercapto can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy, cycloalkylmercapto, heterocycloalkylmercapto, oxo, amino, haloalkyl, hydroxyalkyl, carboxyl or carboxylate group, and the like.

The term “substituted or unsubstituted amino” refers to NH₂, monosubstituted NH₂ and disubstituted NH₂. When substituted, the monosubstituent or disubstituted group is preferably independently selected from alkyl, hydroxyl, mercapto, alkenyl, alkynyl, alkoxy, alkylmercapto, alkylamino, cycloalkyl, heterocycloalkyl, aryl, heteroaryl, cycloalkoxy, heterocycloalkoxy, cycloalkylmercapto, heterocycloalkylmercapto, oxo group, amino, haloalkyl, hydroxyalkyl, carboxyl or carboxylate group, etc. Disubstituted groups can form a nonaromatic cyclic structure together with the nitrogen atom to which they are attached.

The term “aryl” refers to any stable conjugated hydrocarbon ring system group with 6-18 carbon atoms, preferably 6-10 carbon atoms, which can be monocyclic, bicyclic, tricyclic or more cyclic aromatic groups, such as phenyl, naphthyl and anthracene, etc. The aryl ring can be fused to a heteroaryl, heterocycloalkyl or cycloalkyl ring. The aryl group may be substituted or unsubstituted, and when substituted, the substituent group is preferably one or more groups independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkoxy, heterocycloalkoxy and cycloalksulfhydryl.

The term “heteroaryl” refers to an aromatic ring system in which at least one carbon atom in the ring is replaced by a heteroatom selected from N, O or S, preferably a 5- to 7-membered monocyclic moiety or a 7- to 12-membered bicyclic moiety, more preferably a 5- to 6-membered heteroaryl, such as pyrrolyl, imidazolyl, pyridyl, pyrimidinyl, thiazolyl, thienyl, pyrazinyl, triazolyl, tetrazolyl, oxazolyl, indazolyl, and the like. The heteroayl ring can be fused to a ring of aryl, heterocycloalkyl or cycloalkyl. The heteroaryl can be substituted or unsubstituted, and the substituent is preferably one or more than one groups, independently selected from alkyl, halogen, hydroxyl, mercapto, cyano, alkenyl, alkynyl, alkoxy, alkylmercapto, alkylamino, nitro, cycloalkyl, heterocycloalkyl, aryl, heteroaryl, cycloalkyloxy, heterocycloalkyloxy, heterocycloalkyloxy, cycloalkylmercapto, heterocycloalkylmercapto, oxo, amino, haloalkyl, hydroxyalkyl, carboxyl or carboxylate group.

The term “amino protecting group” refers to any group used to protect an amine, as described in “Protecting Groups in Organic Chemistry” (Greene's Protective Groups in Organic Synthesis, Fifth Edition, Chapter 7, Protection for the Amino Group, P895-1193). Typically, such groups are selected from the group consisting of tert-butoxycarbonyl (Boc), benzyl (Bn), 2,4-dimethoxybenzyl (DMB), p-methyl Oxybenzyl (PMB), benzyloxycarbonyl (Cbz), fluorenylmethoxycarbonyl (Fmoc), allyloxycarbonyl (Alloc), unsubstituted or substituted benzyl or benzenesulfonyl (Bs), p-Tosyl (Ts), 2-naphthalenesulfonyl, trimethylsilyl (TMS), trifluoroacetyl (TFA), trityl (Tr), trichloroacetyl (TCA), formyl (CHO), acetyl (Ac), benzoyl (Bz) or tert-butyl (t-Bu).

The term “hydroxyl protecting group” refers to any group used to protect a hydroxyl group, as described in “Protective Groups in Organic Chemistry” (Greene's Protective Groups in Organic Synthesis, Fifth Edition, Chapter 2, Protection for the Hydroxyl Group, including 1,2- and 1,3-Diols, P17-374). Typically, such groups are selected from benzyl (Bn), p-methoxybenzyl (PMB), methoxy methyl ether (MOM), trimethylsilyl ether (TMS), triethylsilyl ether (TES), tert-butyldiphenylsilyl (TBDPS), tert-butyldimethylsilyl (TBS/TBDMS), triisopropylsilyl (TIPS), allyloxycarbonyl (Alloc), 2-(trimethylsilyl) ethoxycarbonylation (Teoc), trityl (Trt), 2,4-Dimethoxybenzyl (DMB), methoxymethyl ether (MOM), acetyl (Ac), benzoyl (Bz), trityl (Tr), 2-tetrahydropyran (THP)), or other similar ester, ether, acetal, and silicon ether protecting groups.

The term “hydroxyl” refers to —OH.

The term “halogen” refers to fluorine, chlorine, bromine or iodine.

The term “nitro” refers to —O₂.

The term “amino” refers to —NH₂.

The term “cyano” refers to —CN.

The term “carboxylic acid” refers to —C(O)OH.

The term “mercapto” or “sulfhydryl” refers to —SH.

The term “carboxylate group” refers to —C(O)O-alkyl, —C(O) O-aryl, or —C(O) O-cycloalkyl, wherein the definitions of alkyl, the aryl, and the cycloalkyl are as defined above.

The term “substituted” means that one or more than one hydrogen or deuterium atoms in the group, preferably 1 to 5 hydrogens or deuterium atoms, are independently substituted by a corresponding number of substituents.

The term “pharmaceutically acceptable salt” refers to a salt that can retain the biological effectiveness of the free base without other toxic and side effects, and can be an acidic salt, a basic salt or an amphoteric salt. Non-limiting examples include, but are not limited to, acidic salts including hydrochloride, hydrobromide, sulfate, pyrosulfate, bisulfate, sulfite, bisulfite, phosphate, monohydrogen phosphate, dihydrogen phosphate, metaphosphate, pyrophosphate, nitrate, acetate, propionate, caprate, octanoate, formate, acrylate, isobutyrate, hexanoate, heptanoate, oxalate, malonate, succinate, suberate, benzoate, methyl benzoate, phthalate, maleate, methanesulfonate, p-toluenesulfonate, benzenesulfonate, (D), L)-tartrate, citrate, maleate, (D), L-)malate, fumarate, stearate, oleate, cinnamate, laurate, glutamate, aspartate, triflate, mandelate, ascorbate, salicylate, and the like. When the compound of the present invention contains acidic groups, pharmaceutically acceptable salts thereof can further include alkali metal salts (e.g., sodium salt or potassium salt), alkaline earth metal salts (e.g., calcium salt or magnesium salt), organic base salts (e.g., alkyl aromatics, amino acids, etc.).

Abbreviations for any protecting groups, amino acids, and other compounds are commonly used and recognized abbreviations, unless otherwise specified, or refer to IUPAC-IUBC Commission on Biochemical Nomenclature (See Biochem. 1972, 11, 942-944).

### DETAILED DESCRIPTION OF THE PREFERRED EMBODIMENT

The following examples further describe the present invention, but these examples should not limit the scope of the present invention.

In the examples of the invention, the experimental methods without specific conditions are generally in accordance with conventional methods and conditions, or in accordance with the conditions recommended by the manufacturers of raw materials or commodities. The reagents without specific sources are conventional reagents purchased from the market.

All compounds of the present invention can be determined by nuclear magnetic resonance (NMR) or mass spectrometry (MS). The NMR shift (δ) is recorded in units of 10⁻⁶ (ppm). The NMR measuring instrument is Bruker AVANCE-400 spectrometer. The deuterated solvents are deuterated chloroform (CDCl₃), deuterated methanol (CD₃OD), deuterium oxide (D₂O) or deuterated dimethyl sulfoxide (DMSO-d₆), and the internal standard is tetramethylsilane (TMS).

Low-resolution mass spectrometry (MS) is determined by Agilent 6120 quadruple LCMS mass spectrometer.

The HPLC purity is determined by Agilent HPLC Agilent 1260/1220 chromatograph (Agilent Zorb Ax BonusRP 3.5 μm×4.6 mm×150 mm or Boston pHlex ODS 4.6 mm×150 mm×3 μm).

The compounds of the present invention and their intermediates can be isolated and purified by conventional preparative HPLC, silica gel plate, column chromatography, or flash column chromatography.

The thin-layer chromatography silica gel plate uses Yantai Huanghai, Yantai Xinnuo Chemical Industry HSGF254, or Qingdao GF254 silica gel plate. The silica gel plate used for thin-layer chromatography (TLC) is 2.5×5 cm, 0.2 mm-0.25 mm, and the thin layer chromatography separation (Prep-TLC) used for purifying products is 1 mm or 0.4 mm-0.5 mm, 20×20 cm.

Column chromatography (silica gel column chromatography) is generally used in sizes of 100-200 mesh or 200-300 mesh or 300-400 mesh.

The flash separator is Agela Technologies MP200, and the column is generally Flash column silica-CS (12 g-330 g).

The preparative HPLC (Prep-HPLC) is Gilson GX-281, and the column model is Welch Ultimate XB-C18 21.2 mm×250 mm×10 μm.

The chiral columns are CHIRALCEL OD-H, OJ-H or CHIRALPAK AD-H, AS-H 4.6 mm×250 mm×5 μm, and the preparation column types are CHIRALCEL OD-H, OJ-H or CHIRALPAK AD-H, AS-H 10 mm×250 mm×5 μm.

The known starting materials of the present invention can be synthesized by methods known in the art, or purchased from suppliers such as Sigma-Aldrich, ACROS, Alaf, TCI, J&K Scientific, energy-chemical, Accela ChemBio, Macklin, Shanghai Titan Scientific Co., Ltd and the like.

Anhydrous solvents such as anhydrous tetrahydrofuran, anhydrous dichloromethane or anhydrous N,N-dimethylacetamide are commercially available from the above chemical companies.

Unless otherwise specified in the examples, the reaction is generally carried out under a nitrogen or argon atmosphere. The nitrogen or argon atmosphere refers to that the reaction flask is connected to a balloon of nitrogen or argon having a volume of about 1 L and subjected to three pumping displacements.

The hydrogen atmosphere means that the reaction flask is connected to a hydrogen balloon having a volume of about 1 L and subjected to three pumping displacements.

The pressurized hydrogenation reaction uses a pressure-resistant sealed glass reaction vessel and is connected to a hydrogen pressure gauge.

In the examples, unless otherwise specified, the reaction temperature is room temperature, and the temperature is 15-25° C.

The reactions in the examples are generally monitored by LCMS or TLC, wherein the LCMS is as described above. The developing solvent system used for TLC is generally: dichloromethane and methanol, petroleum ether and ethyl acetate, dichloromethane and ethyl acetate, petroleum ether and dichloromethane, ethyl acetate and methanol, etc. The volume ratio of the solvent is adjusted according to the polarity of the compound, and a small amount (0.1%- 10%) of base (such as triethylamine or 37% ammonia water, etc.) or acid (such as acetic acid, etc.) can also be added for adjustment.

The compounds can be purified by Prep-TLC, column chromatography or Agela preparation system. The elution solvent system is generally dichloromethane and methanol, petroleum ether and ethyl acetate, dichloromethane and ethyl acetate, petroleum ether and dichloromethane, ethyl acetate and methanol, etc. The volume ratio of the solvent is adjusted according to the polarity of the compound. A small amount (0.1%- 10%) of base (such as triethylamine or 37% ammonia water, etc.) or acid (such as acetic acid, etc.) can also be added for adjustment.

The following abbreviations are used throughout the present invention:
Ac: acetyl protecting group
Cbz: benzyloxycarbonyl protecting group
Fmoc: 9-fluorenylmethoxycarbonyl protecting group
PMB: p-methoxybenzyl protecting group
TBS: tert-butyldimethylsilyl protecting group
Bn: benzyl protecting group
Boc: tert-butoxycarbonyl protecting group
9-BBN: 9-borabicyclo[3.3.1]nonane
K₃PO₄: potassium phosphate
H₂O: water
PdCl₂(dppf): (1,1′-bis(diphenylphosphino)ferrocene)palladium(II) dichloride
Pdz(dba)₃: tris(dibenzylideneacetone)dipalladium
S-Phos: dicyclohexyl(2′,6′-dimethoxy[1,1′-biphenyl]-2-yl)phosphane
Pd(PPh₃)₄: tetrakis(triphenylphosphine)palladium
PEPPSI-IPr: [1,3-bis(2,6-diisopropylphenyl)imidazol-2-ylidene](3-chloropyridyl)palladium(II) dichloride
XantPhos: 4,5-bis(diphenylphosphino)-9,9-dimethylxanthene
DMF: N,N-dimethylformamide
THF: tetrahydrofuran
Boc₂O: di-tert-butyl decarbonate
DIPEA: diisopropylethylamine
DMAP: 4-dimethylaminopyridine
NBS: N-bromosuccinimide
NIS: N-iodosuccinimide
DMSO: dimethyl sulfoxide
NMP: N-methylpyrrolidone
DMA: N,N-dimethylacetamide
DME: ethylene glycol dimethyl ether
DBU: 1,8-diazabicyclo[5.4.0]undec-7-ene
TMEDA: N,N,N′,N′-Tetramethylethylenediamine

The present invention is further illustrated by the following examples, but the invention is not limited to the scope of the described examples.

### Example 1: Preparation of Compound I-1

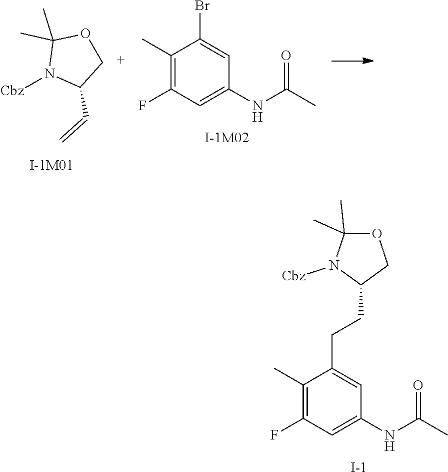

To a solution of compound I-1M01 (prepared according to the synthesis of compound 25 was in J. Org. Chem. 2002, 67, 6, 1802-1815) (1.3 g, 5 mmol) in THF (15 mL) at 0° C. was added 9-BBN (0.5 M in THF, 20 mL, 10 mmol) Under nitrogen atmosphere, and the reaction solution was slowly warmed to room temperature and continues to stir for 2 h. To the above solution was added K₃PO₄ (3 M in H₂O, 3.3 mL, 10 mmol), compound I-1M02 (prepared according to the synthesis method of patent TW201920078A Example 1) (1.1 g, 4.5 mmol) in DMF solution (20 mL) and PdCl₂(dppf) (241 mg, 0.325 mmol). The above mixture was heated to 80° C. and stirred for 16 h. The reaction solution was concentrated, quenched with water, and extracted with ethyl acetate. The organic phases were combined, washed with saturated brine, dried over anhydrous sodium sulfate, filtered, and concentrated under reduced pressure to obtain a crude product, which was separated and purified by silica gel column chromatography to obtain compound I-1 as a foamy solid, (1.87 g, 97% yield).

MS (ESI), m/z, 429.2 [M+1]⁺.

### Examples 2 and 3: Preparation of Compounds I-2 and I-3

According to the synthesis method of Example 1, its chiral isomer compounds I-2 and I-3 are prepared according to raw materials with different configurations:

| | | | MS |
|---|---|---|---|
| Ex- | | | (ESI), |
| amples | raw materials | Structure | m/z |
| Ex- ample 2 | 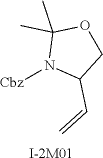 | 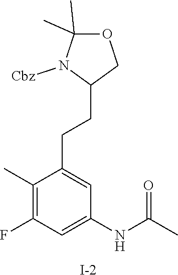 | 429.2 [M + 1]⁺ |
| Ex- ample 3 | 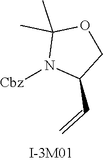 | 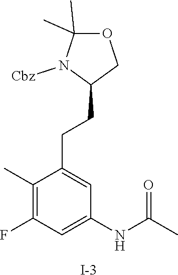 | 429.2 [M + 1]⁺ |

### Example 4: Preparation of Compound I-4

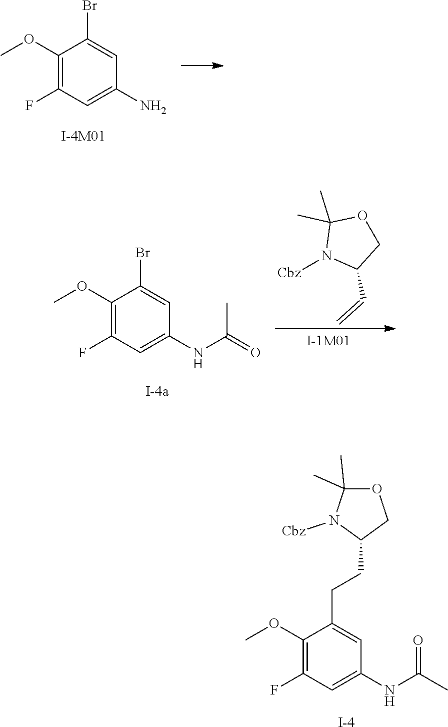

### Preparation of Compound I-4a

To a solution of I-4M01 (3-bromo-5-fluoro-4-methoxyaniline, 10.0 g, 0.05 mol) in dichloromethane (100 mL) was added dropwise acetic anhydride (9.27 g, 0.09 mol), followed by triethylamine (9.18 g, 0.09 mol) under cooling in an ice-water bath and stirred for 12 h. The reaction solution was quenched with water and extracted with dichloromethane. The organic phases were combined, washed with saturated brine, dried over anhydrous sodium sulfate, filtered, and concentrated under reduced pressure to obtain compound I-4a (11 g, 92% yield). MS (ESI), m/z, 261.9 [M+1]⁺.

### Preparation of Compound I-4

To a solution of I-1M01 (1.3 g, 5 mmol) in THF (15 mL) was added 9-BBN (0.5 M in THF, 20 mL, 10 mmol) at 0° C. and nitrogen atmosphere, and the reaction solution slowly warmed to room temperature and continued stirring for 2 h. To the above solution were added K₃PO₄ (3 M in H₂O, 3.3 mL, 10 mmol), compound I-4a (1.3 g, 5.0 mmol) in DMF (20 mL), and PdCl₂(dppf) (241 mg, 0.325 mmol). The above mixture was heated to 80° C. and stirred for 16 h. The reaction solution was concentrated, quenched with water, and extracted with ethyl acetate. The organic phases were combined, washed with saturated brine, dried over anhydrous sodium sulfate, filtered, and concentrated under reduced pressure to obtain a crude product, which was separated and purified by silica gel column chromatography to obtain compound I-4 (1.95 g, yield 88%).

MS (ESI), m/z, 445.2 [M+1]⁺.

### Example 5: Preparation of Compound I-5

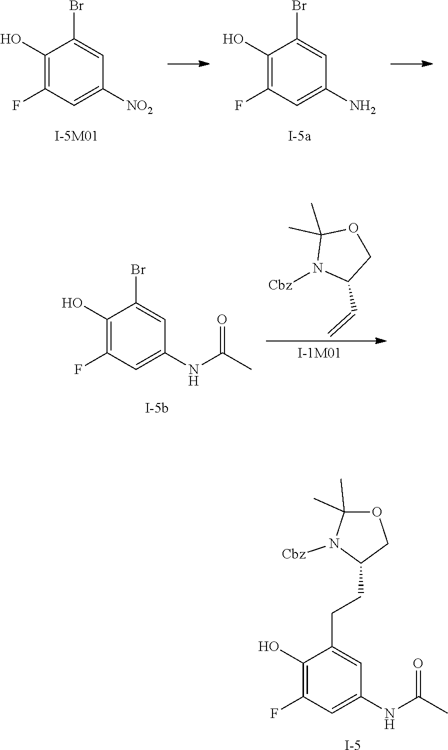

### Preparation of Compound I-5a

To a solution of I-5M01 (2-bromo-6-fluoro-4-nitrophenol, 5.0 g, 0.02 mol) in ethanol (50 mL) at room temperature was added water (10 mL), acetic acid (5 mL), and iron powder (5.9 g, 0.1 mol). The reaction solution was heated to 80° C. for 1 h, filtered, ethyl acetate and water were added to the filtrate, and extracted with ethyl acetate. The organic phases were combined, washed with saturated brine, dried over anhydrous sodium sulfate, filtered, and concentrated under reduced pressure to obtain compound I-5a (4.3 g, 98% yield).

MS (ESI), m/z, 205.9 [M+1]⁺.

### Preparation of Compound I-5b

To a solution of I-5a (3.0 g, 0.015 mol) in dichloromethane (50 mL) was added dropwise acetic anhydride (2.2 g, 0.022 mol) under cooling in an ice-water bath, followed by triethylamine (2.9 g, 0.03 mol), stir for 12 h. The reaction solution was quenched with water, extracted with dichloromethane, the organic phases were combined, washed with saturated brine, dried over anhydrous sodium sulfate, filtered, and concentrated under reduced pressure to obtain a crude product, which was purified by silica gel column chromatography to obtain compound I-5b (2.5 g, 69% yield).

MS (ESI), m/z, 247.9 [M+1]⁺.

### Preparation of Compound I-5

To a solution of I-1M01 (1.3 g, 5 mmol) in THF (15 mL) was added 9-BBN (0.5 M in THF, 20 mL, 10 mmol) at 0° C. and nitrogen atmosphere. The reaction solution slowly warmed to room temperature and continue stirring for 2 h. To the above solution were added K₃PO₄ (3 M in H₂O, 3.3 mL, 10 mmol), compound I-5b (1.2 g, 5.0 mmol) in DMF (20 mL), and PdCl₂(dppf) (241 mg, 0.325 mmol). The above mixture was heated to 80° C. and stirred for 12 hours, concentrated, quenched with water, extracted with ethyl acetate, combined the organic phases, washed with saturated brine, dried over anhydrous sodium sulfate, filtered, concentrated under reduced pressure to obtain a crude product, which was purified by silica gel column chromatography to obtain compound I-5 (1.6 g, yield 75%).

MS (ESI), m/z, 431.1 [M+1]⁺.

### Example 6: Preparation of Compound I-6

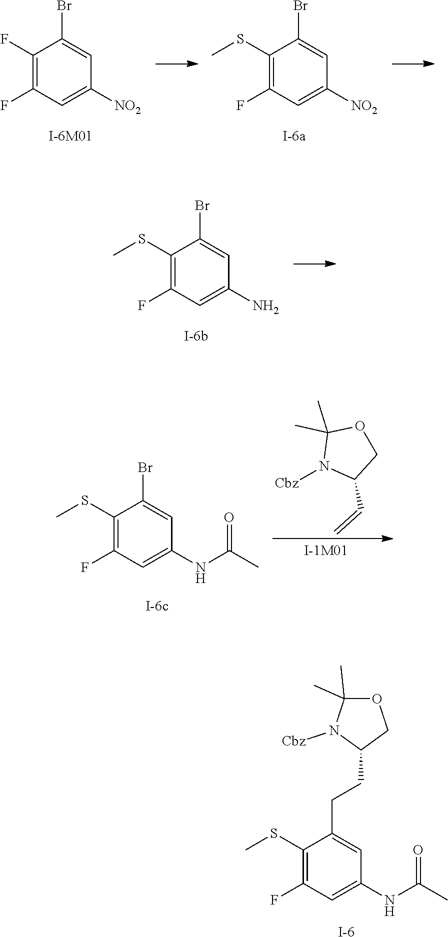

### Preparation of Compound I-6a

To a solution of I-6M01 (2,3-difluoro-5-nitrobromobenzene, 5.0 g, 0.02 mol) in DMF (50 mL) was added sodium thiomethoxide (3.0 g, 0.04 mmol) at 0° C. under nitrogen atmosphere. The reaction solution was warmed to room temperature and stirred for 12 h. The reaction solution was poured into ice water, stirred for 10 minutes, filtered, and the filter cake was washed with water and dried to obtain compound I-6a (4.5 g, yield 80%).

MS (ESI), m/z, 265.9 [M+1]⁺.

### Preparation of Compound I-6b

To a solution of I-6a (4.0 g, 0.015 mol) in ethanol (40 mL) was added water (8 mL), acetic acid (5 mL), and iron powder (4.2 g, 0.08 mol) at room temperature. The reaction solution was heated to 80° C. for 1 h, filtered, ethyl acetate and water were added to the filtrate, and extracted with ethyl acetate. The organic phases were combined, washed with saturated brine, dried over anhydrous sodium sulfate, filtered, and concentrated under reduced pressure to obtain compound I-6b (3.1 g, 86% yield).

MS (ESI), m/z, 235.9 [M+1]⁺.

### Preparation of Compound I-6c

To a solution of I-6b (1.0 g, 4.3 mmol) in dichloromethane (20 mL) was added dropwise acetic anhydride (870 mg, 8.5 mmol) and triethylamine (864 mg, 8.5 mmol) at room temperature, and stirred for 1 h. The reaction solution was quenched with water, extracted with dichloromethane, the organic phases were combined, washed with saturated brine, dried over anhydrous sodium sulfate, filtered, and concentrated under reduced pressure to obtain compound I-6c (1.1 g, 92% yield).

MS (ESI), m/z, 278.1 [M+1]⁺.

### Preparation of Compound I-6

To a solution of I-1M01 (800 mg, 3.0 mmol) in THF (10 mL) was added 9-BBN (0.5 M in THF, 12 mL, 6.1 mmol) at 0° C. under nitrogen atmosphere. Then the reaction solution was warmed to room temperature and stir for 2 h. To the above solution were added K₃PO₄ (1.3 g dissolved in 3 mL water, 6.1 mmol), compound I-6c (852 mg, 3.1 mmol) in DMF (10 mL) and PdCl₂(dppf) (224 mg, 0.31 mmol). The above mixture was heated to 80° C. and stirred for 12 hours, concentrated, quenched with water, extracted with ethyl acetate, combined organic phases, washed with saturated brine, dried over anhydrous sodium sulfate, filtered, and concentrated under reduced pressure to obtain a crude product, which was purified by silica gel column chromatography to compound I-6 (1.1 g, yield 78%).

MS (ESI), m/z, 461.1 [M+1]⁺.

### Example 7: Preparation of Compound I-7

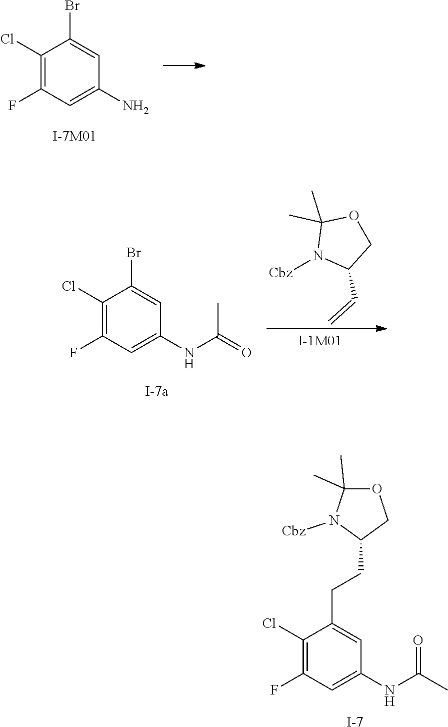

### Preparation of Compound I-7a

To a solution of I-7M01 (prepared according to the synthesis method of the fragment of compound 58 in patent US2019177338 A1) (1.0 g, 4.5 mmol) in dichloromethane (20 mL) was added dropwise acetic anhydride (0.91 g, 9.0 mmol) and triethylamine (0.91 g, 9.0 mmol) at room temperature and stirred for 1 h. The reaction solution was quenched with water, extracted with dichloromethane, the organic phases were combined, washed with saturated brine, dried over anhydrous sodium sulfate, filtered, and concentrated under reduced pressure to obtain compound I-7a (1.1 g, 92% yield).

MS (ESI), m/z, 266.1 [M+1]⁺.

### Preparation of Compound I-7

To a solution of I-1M01 (1.0 g, 3.8 mmol) in THF (10 mL) was added 9-BBN (0.5 M in THF, 15 mL, 7.7 mmol) at 0° C. under nitrogen atmosphere. The reaction solution was slowly warmed to room temperature and stirred for 2 h. To the above solution were added K₃PO₄ (1.6 g dissolved in 3.5 mL water, 3.8 mmol), compound I-7a (1.0 g, 3.8 mmol) in DMF (10 mL) and PdCl₂(dppf) (280 mg, 0.38 mmol). The above mixture was heated to 80° C. and stirred for 12 hours, concentrated, quenched with water, extracted with ethyl acetate, combined organic phases, washed with saturated sodium chloride solution, dried over anhydrous sodium sulfate, filtered, and concentrated under reduced pressure to obtain a crude product, which was purified by silica gel column chromatography to obtain compound I-7 (1.5 g, yield 87%).

MS (ESI), m/z, 449.1 [M+1]⁺.

### Example 8: Preparation of Compound I-8

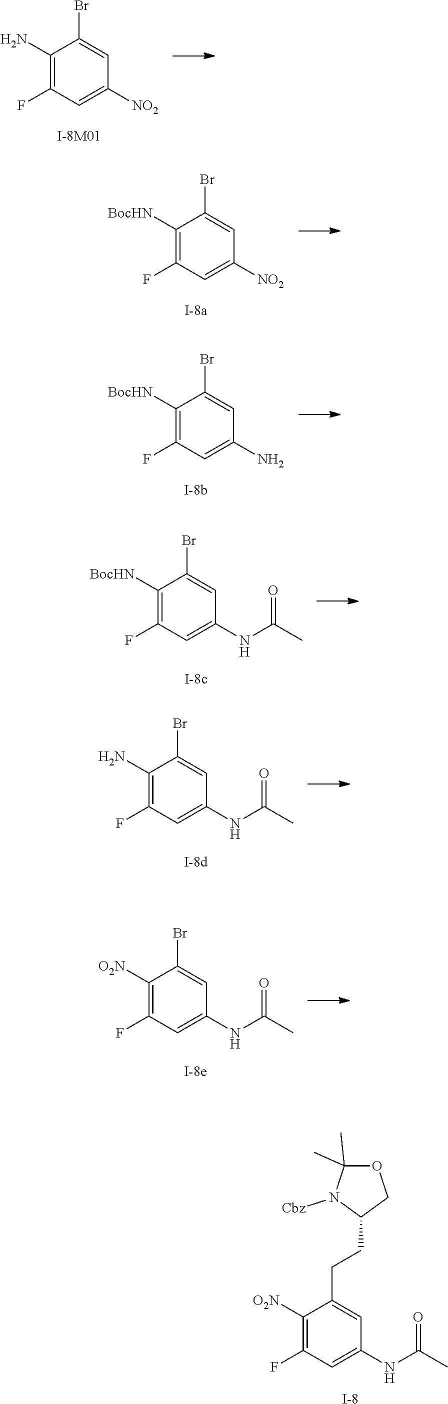

### Preparation of Compound I-8a

To a solution of I-8M01 (5.0 g, 0.02 mol) in THF (50 mL) was added Boc₂O (9.3 g, 0.04 mol) and DMAP (1.3 g, 0.01 mol). The reaction solution was heated to reflux for 1 h, concentrated, diluted with water, extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-8a, 6.1 g, yield 86%. MS (ESI), m/z, 335.0 [M+1]⁺.

### Preparation of Compound I-8b

To a solution of I-8a (3.0 g, 0.01 mol) in ethanol (30 mL) and water (10 mL) was added ammonium chloride (4.8 g, 0.09 mol) and Zn powder (5.0 g, 0.09 mol). The above mixture was heated to 90° C. and stirred for 2 h, filtered, and the filtrate was diluted with water and extracted with ethyl acetate. The organic phases were combined, washed with saturated sodium chloride solution, dried over anhydrous sodium sulfate, filtered, and concentrated under reduced pressure to obtain a crude product, which was separated and purified by silica gel column chromatography to obtain compound I-8b (2.2 g, yield 80%).

MS (ESI), m/z, 305.1 [M+1]⁺.

### Preparation of Compound I-8c

To a solution of I-8b (2.0 g, 6.6 mmol) in dichloromethane (40 mL) was added dropwise acetic anhydride (1.3 g, 13.1 mmol) and triethylamine (1.3 g, 13.1 mmol) at room temperature and stirred for 1 h. The reaction solution was quenched with water and extracted with dichloromethane. The organic phases were combined, washed with saturated sodium chloride solution, dried over anhydrous sodium sulfate, filtered, and concentrated under reduced pressure to obtain a crude product, which was purified by silica gel column chromatography to obtain compound I-8c (2.1 g, yield 92%).

MS (ESI), m/z, 347.1 [M+1]⁺;

### Preparation of Compound I-8d

To a solution of I-8c (2.0 g, 5.8 mmol) in dichloromethane (20 mL) was added trifluoroacetic acid (4 mL) and stirred for 1 h at room temperature. The mixture was concentrated under reduced pressure, diluted with water, adjusted with 2N sodium hydroxide solution, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain the target product I-8d (1.2 g, yield 84%).

MS (ESI), m/z, 246.9 [M+1]⁺.

### Preparation of Compound I-8e

A mixture of sodium perborate tetrahydrate (3.1 g, 20.2 mmol) and acetic acid (20 mL) was heated to 80° C. Under stirring, a solution of compound I-8d (1.0 g, 4.0 mmol) in acetic acid (5 mL) was slowly added. After the addition, the reaction solution was heated and stirred for 2 h. The reaction solution was cooled to room temperature, poured into ice water, and extracted with ethyl acetate. The organic phases were combined, washed with saturated sodium bicarbonate, washed with saturated sodium chloride solution, dried over anhydrous sodium sulfate, filtered, and concentrated under reduced pressure to obtain a crude product, which was separated and purified by silica gel column chromatography to obtain the target product I-8e (800 mg, collected rate 71%).

MS (ESI), m/z, 276.9 [M+1]⁺.

### Preparation of Compound I-8

To a solution of I-1M01 (1.9 g, 7.2 mmol) in THF (30 mL) was added 9-BBN (0.5 M in THF, 30 mL, 14.4 mmol) at 0° C. and nitrogen atmosphere, and the reaction solution was slowly warmed to room temperature. and stirred for 2 h. To the above solution were added K₃PO₄ (3.1 g dissolved in 5 mL water, 14.4 mmol), compound I-8e (2.0 g, 7.2 mmol) in DMF (30 mL) and PdCl₂(dppf) (264 mg, 0.36 mmol). The above mixture was heated to 80° C. and stirred for 24 h, concentrated, quenched with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-8 (1.7 g, yield 51%).

MS (ESI), m/z, 460.2 [M+1]⁺.

### Example 9: Preparation of Compound I-9

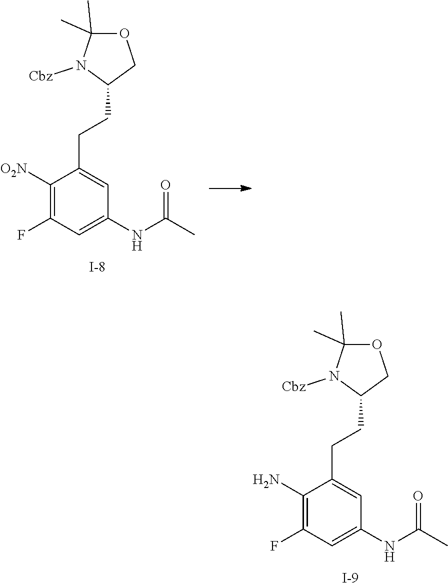

To a solution of I-8 (1.0 g, 2.18 mmol) in ethanol (10 mL) and water (3 mL) was added ammonium chloride (1.2 g, 21.8 mmol, 10.00 eq) and Zn powder (1.2 g, 21.8 mmol, 10.00 eq). The above mixture was heated to 85° C. and stirred for 4 hours, filtered, diluted with ethyl acetate, washed with water, washed with saturated sodium chloride solution, dried over anhydrous sodium sulfate, filtered to remove the desiccant, concentrated under reduced pressure to obtain a crude product, and purified by silica gel column chromatography to obtain compound I-9 (0.75 g, yield 80%).

MS (ESI), m/z, 430.1 [M+1]⁺.

### Example 10: Preparation of Compound I-10

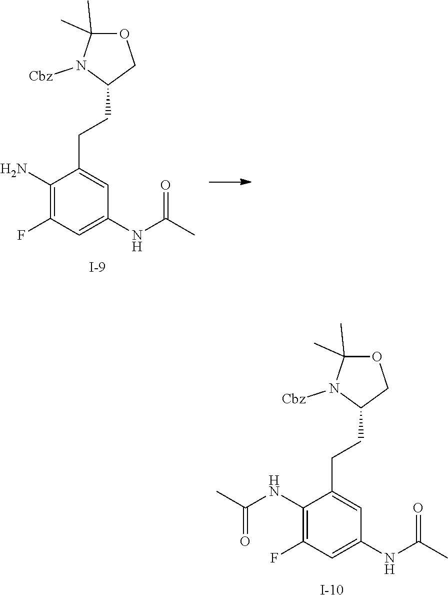

To a solution of I-9 (500 mg, 1.2 mmol) in dichloromethane (20 mL) was added dropwise acetic anhydride (238 mg, 2.3 mmol) in an ice-water bath, followed by triethylamine (235 mg, 2.3 mmol). The reaction solution was warmed to room temperature and stirred for 12 h. The reaction solution was quenched with water, extracted with dichloromethane. The organic phases were combined, washed with saturated sodium chloride solution, dried over anhydrous sodium sulfate, filtered, and concentrated under reduced pressure to obtain compound I-10 (510 mg, 92% yield).

MS (ESI), m/z, 472.1 [M+1]⁺.

### Example 11: Preparation of Compound I-11

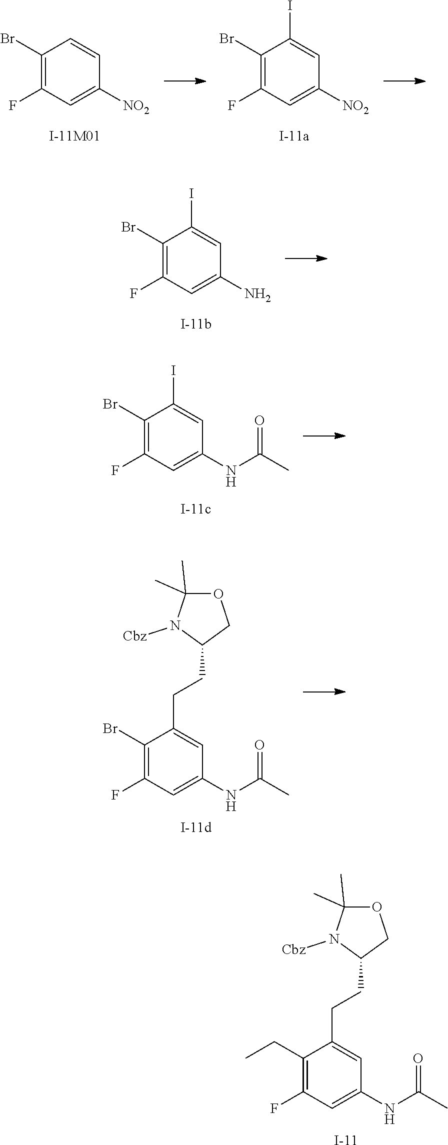

### Preparation of Compound I-11a

To a solution of I-11M01 (110 g, 0.5 mol) in concentrated sulfuric acid (500 mL) was added iodine (51 g, 0.2 mol) and sodium iodate (39.6 g, 0.2 mol) in an ice-water bath. The reaction solution was slowly heated to 25° C. and stirred for 48 h. The reaction solution was added to sodium sulfite solution to quench in an ice bath, stirred for 30 minutes, filtered, and the filter cake was recrystallized with ethanol twice to obtain compound I-11a (55 g, yield 32%).

¹H NMR (400 MHz, CDCl₃) δ 8.54 (dd, J=2.3, 1.6 Hz, 1H), 7.97 (dd, J=7.6, 2.5 Hz, 1H).

### Preparation of Compound I-11b

To a solution of I-11a (25 g, 72.3 mmol) in ethanol (500 mL) was added concentrated hydrochloric acid (85 mL) and tin (II) chloride dihydrate (65 g, 287 mmol) in an ice-water bath. The reaction solution was warmed to room temperature and stirred for 16 hours, and the reaction was monitored by LCMS. After the reaction solution was concentrated, water was added, adjusted to pH=10−12 with 2 N NaOH, filtered, and the filtrate was extracted with ethyl acetate. The organic phases were combined, washed with saturated sodium chloride solution, dried over anhydrous sodium sulfate, filtered, and concentrated under reduced pressure to obtain compound I-11b (21.3 g, yield 93%).

MS (ESI), m/z, 315.8 [M+1]⁺.

### Preparation of Compound I-11c

To a solution of I-11b (21.3 g, 67.4 mmol) in dichloromethane (200 mL) was added triethylamine (13.6 g, 135 mol). The mixture was cooled to −20-10° C. and acetyl chloride (7.94 g, 101 mmol) was added dropwise. After addition complete, the mixture was stirred at 0° C. for 30 minutes, and the reaction was monitored by LCMS. The reaction was quenched with ice water and extracted with dichloromethane. The organic phases were combined, washed with saturated sodium chloride aqueous solution, dried over anhydrous sodium sulfate, concentrated, and filtered to obtain solid product I-11c (20 g, yield 83%).

MS (ESI), m/z, 357.8 [M+1]⁺.

### Preparation of Compound I-11d

To a solution of I-1M01 (1.0 g, 3.8 mmol) in THF (20 mL) was added 9-BBN (0.5 M in THF, 15 mL, 7.7 mmol) at 0° C. under nitrogen atmosphere, and the reaction solution slowly warmed to room temperature and continue stirring for 2 h. To the above solution were added K₃PO₄ (1.6 g dissolved in 5 mL water, 7.7 mmol), compound I-11c (1.4 g, 3.8 mmol) in DMF (10 mL) and PdCl₂(dppf) (140 mg, 0.19 mmol). The above mixture was heated to 80° C. and stirred for 10 h, concentrated, quenched with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound 1-11d (1.6 g, yield 85%).

MS (ESI), m/z, 493.2 [M+1]⁺.

### Preparation of Compound I-11

To a solution of I-11d (1.0 g, 2.0 mmol) in dioxane (20 mL) was added ethylboronic acid (0.45 g, 6.1 mmol), K₃PO₄ (1.1 g, 4.1 mmol), Pd₂(dba)₃ (92 mg, 0.1 mmol), S-Phos (83 mg, 0.2 mmol), and water (1 mL). The above reaction solution was heated to 100° C. under nitrogen atmosphere and stirred for 12 h. The reaction solution was filtered, water was added to the filtrate and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-11 (750 mg, yield 83%). MS (ESI), m/z, 443.2 [M+1]⁺.

### Example 12: Preparation of Compound I-12

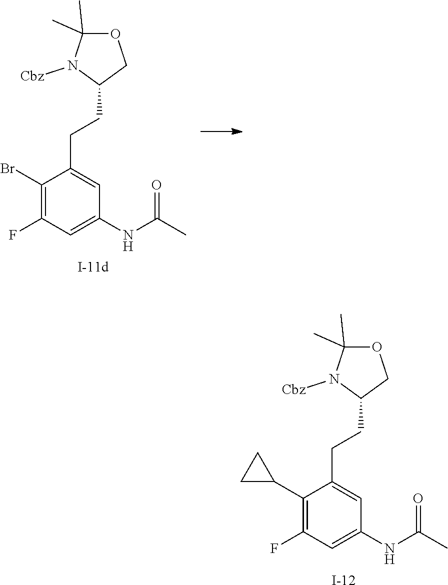

To a solution of I-11d (1.0 g, 2.0 mmol) in dioxane (20 mL) was added cyclopropylboronic acid (0.52 g, 6.1 mmol), K₃PO₄ (1.1 g, 4.1 mmol), and Pd₂(dba)₃ were added (92 mg, 0.1 mmol), S-Phos (83 mg, 0.2 mmol), and water (1 mL). The above reaction solution was heated to 100° C. under nitrogen atmosphere and stirred for 12 h. The reaction solution was filtered, water was added to the filtrate and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-12 (710 mg, yield 77%).

MS (ESI), m/z, 455.2 [M+1]⁺.

### Example 13: Preparation of Compound I-13

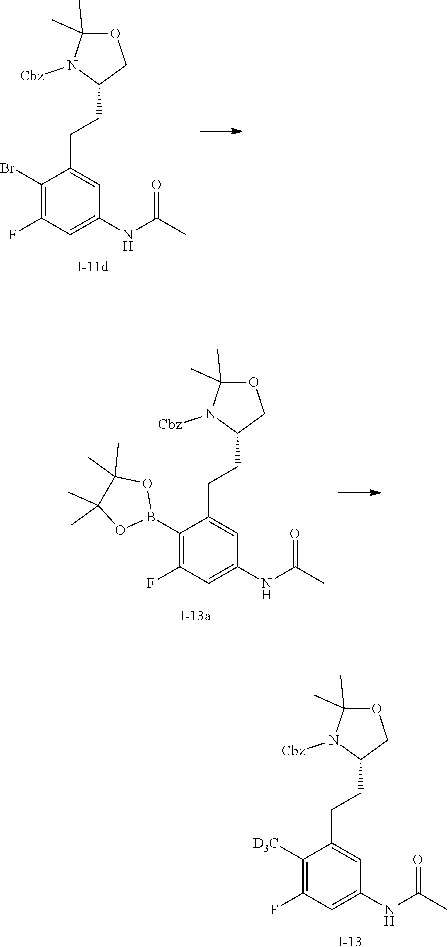

### Preparation of Compound I-13a

To a solution of 1-11d (2.0 g, 4.06 mmol) in DMSO (20 mL) was added pinacol diborate (2.1 g, 8.1 mmol), Pd₂(dba)₃ (186 mg, 0.2 mmol), tricyclohexyl phosphine (114 mg, 0.4 mmol) and potassium acetate (0.8 g, 8.1 mmol), stirred at 90° C. for 18 h under nitrogen atmosphere, and the reaction was monitored by LCMS. The reaction solution was cooled to room temperature, filtered, and water was added, and the aqueous phase was extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound 1-13a (1.4 g, yield 63%).

MS (ESI), m/z, 541.2 [M+1]⁺.

### Preparation of Compound I-13

To a solution of I-13a (1.0 g, 1.85 mmol) in DMF and H₂O (20/2 mL) was added deuterated methyl iodide (1.3 g, 9.3 mmol), Pd₂(dba)₃ (85 mg, 0.09 mmol), tris(o-methylphenyl) phosphorus (56 mg, 0.18 mmol), and potassium carbonate (0.77 g, 5.6 mmol). The mixture was stirred at 70° C. for 16 h under nitrogen atmosphere, and the reaction was monitored by LCMS. The reaction solution was filtered, water was added to the filtrate and extracted with ethyl acetate. The organic phases were combined, washed with saturated sodium chloride solution, dried over anhydrous sodium sulfate, filtered, concentrated under reduced pressure to obtain a crude product, which was purified by silica gel column chromatography to obtain compound I-13,540 mg, yield 67%.

MS (ESI), m/z, 432.2 [M+1]⁺.

### Example 14: Preparation of Compound I-14

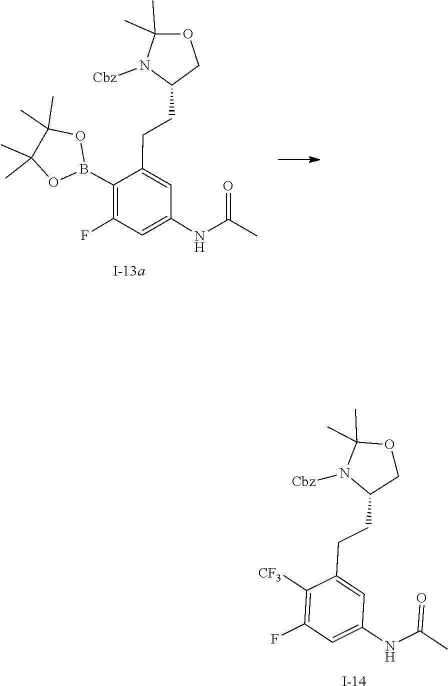

To a solution of I-13a (1.0 g, 1.85 mmol) in DMF (20/2 mL) was added (1,10-phenanthroline) (trifluoromethyl) copper (I) (1.2 g, 3.7 mmol) and potassium fluoride (0.22 g, 3.7 mmol), the mixture was bubbled with air (air balloon) for 10 minutes, the balloon was removed and the reaction solution was heated to 50° C. for 20 h. The reaction solution was diluted with ethyl acetate and filtered. Water was added to the filtrate and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-14 (420 mg, yield 47%).

MS (ESI), m/z, 483.2 [M+1]⁺.

### Example 15. Preparation of Compound I-15

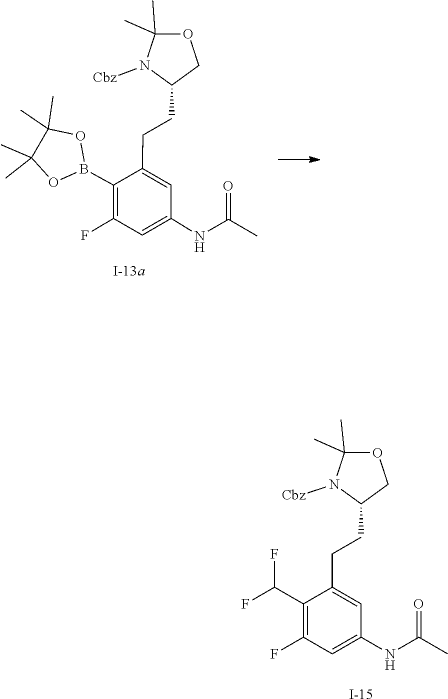

To a solution of I-13a (1.0 g, 1.85 mmol) in dioxane and H₂O (20/2 mL) was added difluoroiodomethane (0.66 g, 3.7 mmol), Pd₂(dba)₃ (85 mg, 0.09 mmol), xantPhos (107 mg, 0.18 mmol), and potassium phosphate (1.0 g, 5.6 mmol). The reaction solution was backfilled with nitrogen and stirred at 90° C. for 20 h, and the reaction was monitored by LCMS. The reaction solution was filtered, water was added to the filtrate and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-15 (650 mg, yield 75%).

MS (ESI), m/z, 465.2 [M+1]⁺.

### Example 16: Preparation of Compound I-16

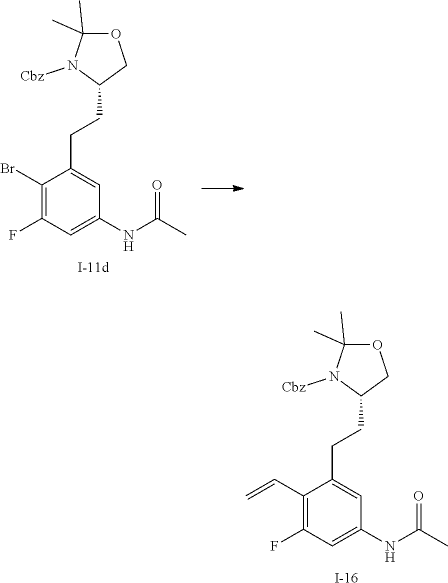

To a solution of compound I-11d (500 mg, 1.02 mmol) in DMF (10 mL) was added tributylvinyltin (644 mg, 2.03 mmol), cesium fluoride (85 mg, 2.03 mmol), and Pd(PPh₃)₄ (117 mg, 0.1 mmol), and heated to 80° C. under nitrogen atmosphere and stirred for 12 h. The reaction solution was filtered, the filtrate was diluted with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-16 (260 mg, yield 58%).

MS (ESI), m/z, 441.1 [M+1]⁺.

### Example 17: Preparation of Compound I-17

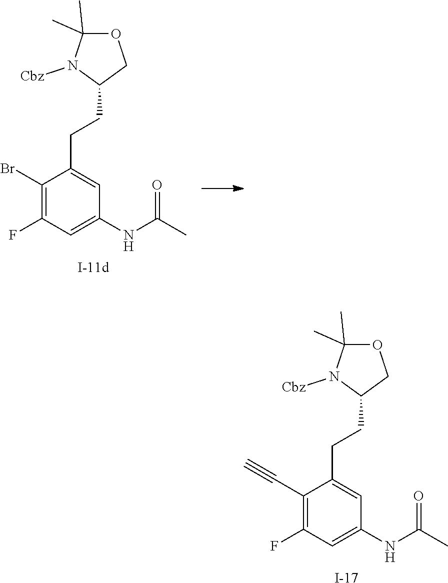

To a solution of I-11d (500 mg, 1.02 mmol) in tetrahydrofuran (10 mL) were added trimethylsilyl acetylene (199 mg, 2.03 mmol), bistriphenylphosphine palladium dichloride (71 mg, 0.1 mmol), copper iodide (19 mg, 0.0 mmol) and triethylamine (205 mg, 2.03 mmol) and heated to 80° C. and stirred for 12 h under nitrogen atmosphere. The reaction solution was filtered, and the filtrate was diluted with water and extracted with ethyl acetate. The organic phases were combined, washed with salt, dried over anhydrous sodium sulfate, and concentrated under reduced pressure. The crude product was directly dissolved in methanol (50 mL), potassium carbonate (280 mg, 2.02 mmol) was added and stirred at room temperature for 2 h. The mixture was filtered, concentrated, and purified by silica gel column chromatography to obtain compound I-17 (270 mg, yield 60%).

MS (ESI), m/z, 439.2 [M+1]⁺.

### Example 18: Preparation of Compound I-18

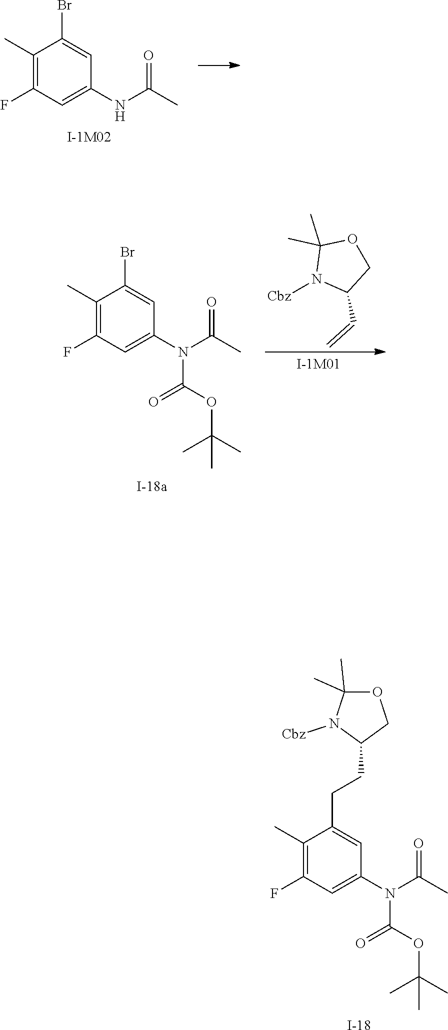

### Preparation of Compound I-18a

To a solution of I-1M02 (1.0 g, 4.08 mmol) in dichloromethane (15 mL) were added (Boc)₂O (1.3 g, 6.1 mmol) and DMAP (598 mg, 4.90 mmol) and the reaction was continued for 12 h at room temperature. The above reaction solution was quenched by water and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-18a (1.2 g, yield 85%).

MS (ESI), m/z, 346.0 [M+1]⁺.

### Preparation of Compound I-18

To a solution of I-1M01 (1.0 g, 3.8 mmol) in THF (20 mL) was added 9-BBN (0.5 M in THF, 15 mL, 7.7 mmol) at 0° C. and under nitrogen atmosphere. The reaction solution slowly warmed to room temperature and stirred for 2 h. To the above solution were added K₃PO₄ (1.6 g dissolved in 5 mL water, 7.7 mmol), compound I-18a (1.3 g, 3.8 mmol) in DMF (10 mL) and PdCl₂(dppf) (140 mg, 0.19 mmol). The above mixture was heated to 80° C. and stirred for 8 h, concentrated, quenched with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-18 (1.9 g, yield 88%). MS (ESI), m/z, 529.2 [M+1]⁺.

### Example 19: Preparation of Compound I-19

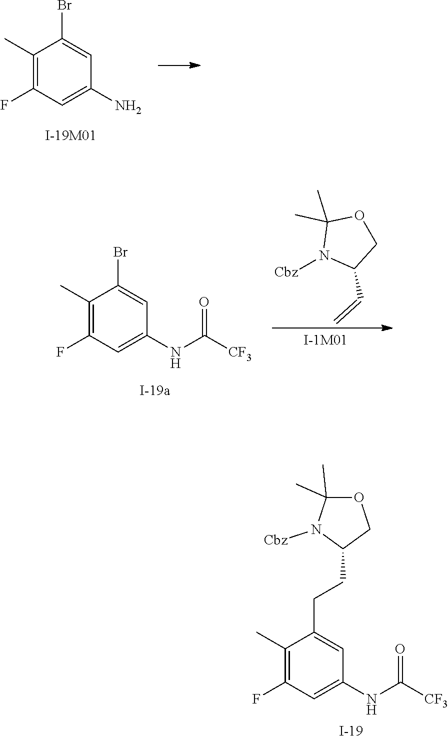

### Preparation of Compound I-19a

To the compound 3-bromo-5-fluoro-4-methylaniline (1.0 g, 4.9 mmol) in methanol (20 mL) was added triethylamine (1.0 g, 9.9 mmol) at 0° C., followed by ethyl trifluoroacetate (1.4 g, 9.9 mmol). The reaction solution was warmed to room temperature and continued to stir for 12 h, concentrated, added water, and extracted with ethyl acetate. The organic phases were combined, washed with saturated sodium chloride solution, dried over anhydrous sodium sulfate, filtered, and concentrated under reduced pressure to obtain compound I-19a (1.4 g, yield 94%).

MS (ESI), m/z, 299.9 [M+1]⁺.

### Preparation of Compound I-19

To a solution of I-1M01 (1.0 g, 3.8 mmol) in THF (20 mL) was added 9-BBN (0.5 M in THE, 15 mL, 7.7 mmol) at 0° C. under nitrogen atmosphere. The reaction solution was slowly warmed to room temperature and continued to stir for 2 h. To the above solution were added K₃PO₄ (1.6 g dissolved in 5 mL water, 7.7 mmol), compound I-19a (1.1 g, 3.8 mmol) in DMF (10 mL) and PdCl₂(dppf) (140 mg, 0.19 mmol). The above mixture was heated to 80° C. and stirred for 20 h, concentrated, quenched with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-19 (1.5 g, yield 81%).

MS (ESI), m/z, 483.1 [M+1]⁺.

### Examples 20-24: Preparation of Compounds I-20 to I-24

According to the synthetic method of Example 19, compounds I-20 to I-24 were prepared according to their respective raw materials:

| Examples | raw materials | Structure | MS (ESI), m/z |
|---|---|---|---|
| Example 20 | 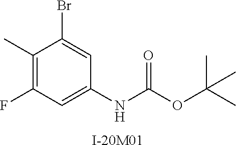 | 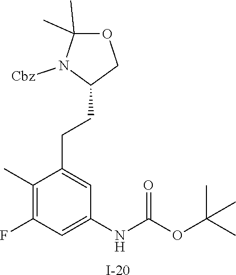 | 487.1 [M + 1]⁺ |
| Example 21 | 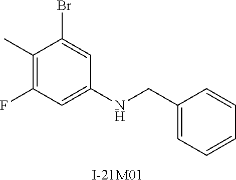 | 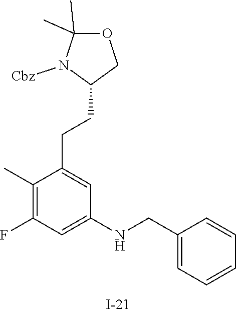 | 477.2 [M + 1]⁺ |
| Example 22 | 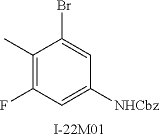 | 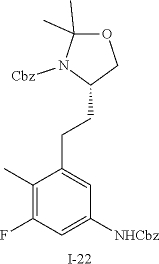 | 521.2 [M + 1]⁺ |
| Example 23 | 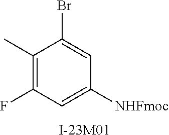 | 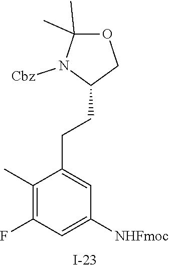 | 609.1 [M + 1]⁺ |
| Example 24 | 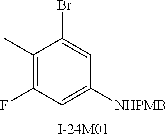 | 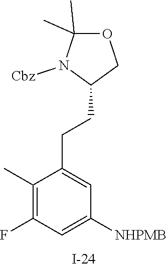 | 507.2 [M + 1]⁺ |

### Example 25: Preparation of Compound I-20

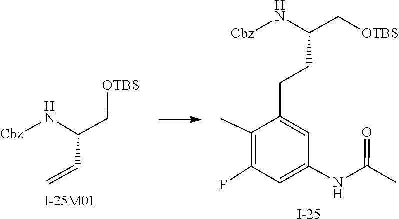

To a solution of I-25M01 (prepared according to the synthesis method of compound 9 in the literature Chem. Eur. J. 2004, 10, 544-553) (1.0 g, 3.0 mmol) in THF (20 mL) was added 9-BBN (0.5 M in THF, 12 mL, 8.1 mmol) at 0° C. under nitrogen atmosphere. The reaction solution was slowly warmed to room temperature and continued to stir for 2 h. To the above solution were added K₃PO₄ (1.7 g dissolved in 5 mL water, 6.0 mmol), compound I-M02 (0.7 g, 3.0 mmol) in DMF (10 mL) and PdCl₂(dppf) (109 mg, 0.15 mmol). The above mixture was heated to 80° C. and stirred for 12 h, concentrated, quenched with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-25, 1.1 g, with a yield of 73%.

MS (ESI), m/z, 503.1 [M+1]⁺.

### Examples 26 and 27: Preparation of Compounds I-26 to I-27

According to the synthetic method of Example 25, compounds I-26 and I-27 were prepared according to their respective starting materials.

| Examples | raw materials | Structure | MS (ESI), m/z |
|---|---|---|---|
| Example 26 | 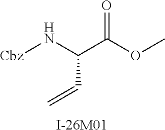 | 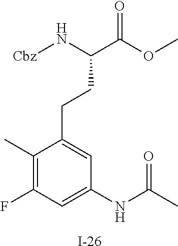 | 417.2 [M + 1]⁺ |
| Example 27 | 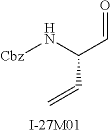 | 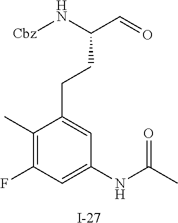 | 387.3 [M + 1]⁺ |

### Example 28: Preparation of Compound I-28

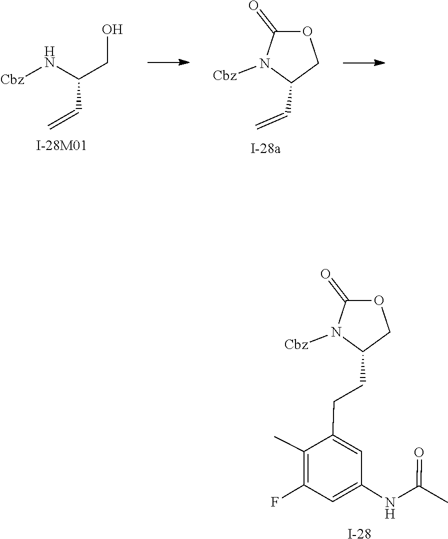

### Preparation of Compound I-28a

To a solution of I-28M01 (prepared according to the synthesis method of compound 1a in Tetrahedron. Letters. 2010, 51, 3226-3228) (1.0 g, 4.5 mmol) in dry dichloromethane (20 mL) was added triphosgene in dichloromethane solution (1.0 g, 6.8 mmol, 5 mL) and DIPEA (1.2 g, 9.05 mmol) at 0° C. The temperature of the reaction solution was warmed to room temperature and stirred for 12 h. The reaction solution was quenched with water and extracted with dichloromethane. The organic phases were combined, washed with saturated sodium chloride solution, dried over anhydrous sodium sulfate, filtered, and concentrated under reduced pressure to obtain a crude product, which was purified by silica gel column chromatography to obtain compound I-28a (1.0 g, yield 89%).

MS (ESI), m/z, 248.1 [M+1]⁺.

### Preparation of Compound I-28

To a solution of I-28a (1.0 g, 4.1 mmol) in THF (20 mL) was added 9-BBN (0.5 M in THF, 16 mL, 8.1 mmol) at 0° C. under nitrogen atmosphere. The reaction solution slowly warmed to room temperature and continued to stir for 2 h. To the above solution were added K₃PO₄ (2.3 g dissolved in 5 mL water, 8.1 mmol), compound I-M02 (1.0 g, 4.1 mmol) in DMF (10 mL) and PdCl₂(dppf) (148 mg, 0.2 mmol). The above mixture was heated to 80° C. and stirred for 12 h, concentrated, quenched with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel chromatography column to obtain compound I-28 (1.3 g, yield 77%).

MS (ESI), m/z, 415.1 [M+1]⁺.

### Examples 29-37: Preparation of Compounds I-29 to I-37

According to the synthetic method of Example 28, compounds I-29 to I-37 were prepared according to their respective raw materials:

| Examples | raw materials | Structure | MS (ESI), m/z |
|---|---|---|---|
| Example 29 | 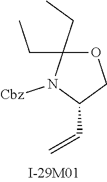 | 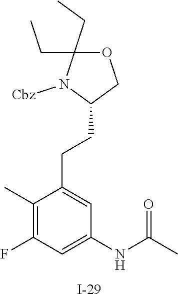 | 457.2 [M + 1]⁺ |
| Example 30 | 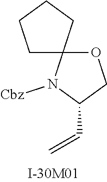 | 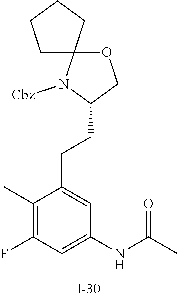 | 455.1 [M + 1]⁺ |
| Example 31 | 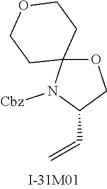 | 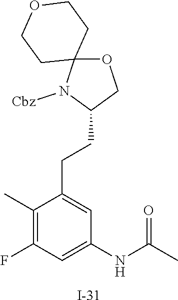 | 471.1 [M + 1]⁺ |
| Example 32 | 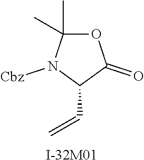 | 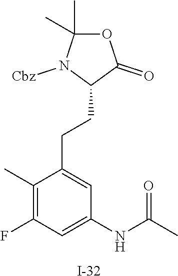 | 443.2 [M + 1]⁺ |
| Example 33 | 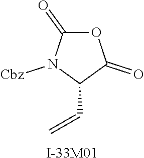 | 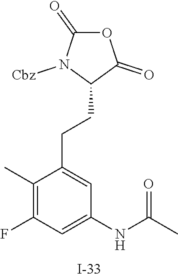 | 429.2 [M + 1]⁺ |
| Example 34 | 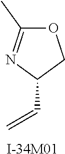 | 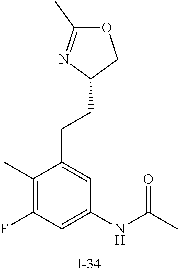 | 279.2 [M + 1]⁺ |
| Example 35 | 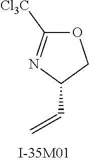 | 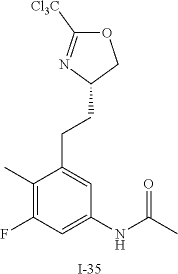 | 381.1 [M + 1]⁺ |
| Example 36 | 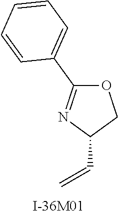 | 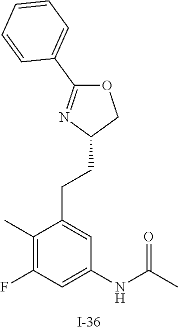 | 341.0 [M + 1]⁺ |
| Example 37 | 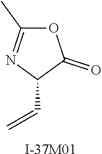 | 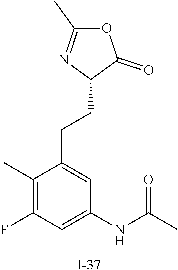 | 293.2 [M + 1]⁺ |

### Example 38: Preparation of Compound I-38

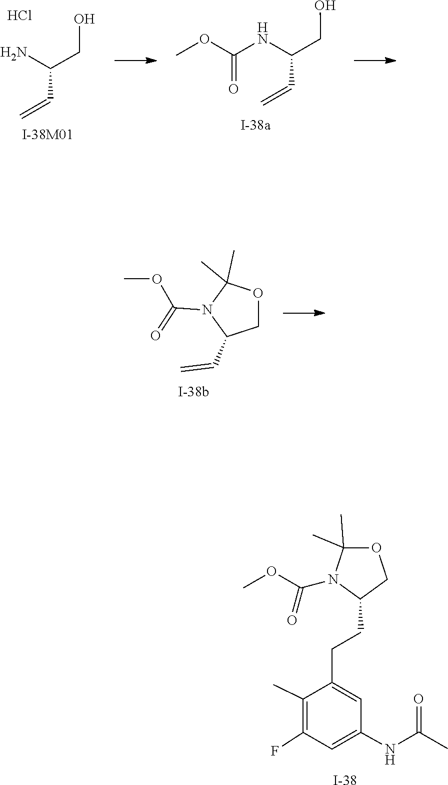

### Preparation of Compound I-38a

To a mixed solution of I-38M01 (prepared by the synthesis method of compound 1 in Synthesis. 1998, 1707-1709) (1.0 g, 4.5 mmol) in tetrahydrofuran and water (10/10 mL) was added sodium bicarbonate (2.6 g, 24.4 mmol) at 0° C., followed by dropwise addition of methyl chloroformate (0.9 g, 9.8 mmol). The reaction solution was warmed to room temperature and stirred for 2 h, diluted with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel chromatography column to obtain compound I-38a (1.1 g, yield 93%). MS (ESI), m/z, 146.1 [M+1]⁺.

### Preparation of Compound I-38b

To a solution of I-38a (1.1 g, 7.6 mmol) in acetone (100 mL) were added 2,2-dimethoxypropane (7.9 g, 75.8 mmol) and boron trifluoride ether (0.54 g, 3.8 mmol). The above reaction solution was stirred for 2 h at room temperature and under nitrogen atmosphere, then added triethylamine (0.5 mL) to quench, concentrated, and purified by silica gel column chromatography to obtain compound I-38b (1.2 g, yield 85%). MS (ESI), m/z, 186.2 [M+1]⁺.

### Preparation of Compound I-38

To a solution of I-38a (1.0 g, 5.4 mmol) in THF (40 mL) was added 9-BBN (0.5 M in THF, 22 mL, 10.8 mmol) at 0° C. under nitrogen atmosphere. The reaction solution was slowly warmed to room temperature and continued to stir for 2 h. To the above solution was added K₃PO₄ (3.0 g dissolved in 10 mL water, 10.8 mmol), I-M02 (1.3 g, 5.4 mmol) in DMF solution (10 mL) and PdCl₂(dppf) (197 mg, 0.27 mmol). The above mixture was heated to 90° C. and stirred for 12 h, concentrated, quenched with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel chromatography column to obtain compound I-38 (1.4 g, yield 73%).

MS (ESI), m/z, 353.2 [M+1]⁺.

### Examples 39-45: Preparation of Compounds I-39 to I-45

According to the synthetic method of Example 38, compounds I-39 to I-45 were prepared according to their respective raw materials:

| Examples | raw materials | Structure | MS (ESI), m/z |
|---|---|---|---|
| Example 39 | 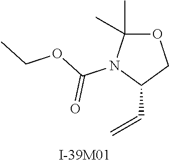 | 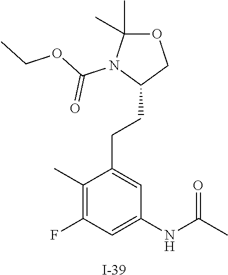 | 367.2 [M + 1]⁺ |
| Example 40 | 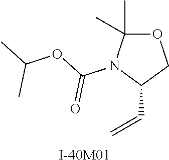 | 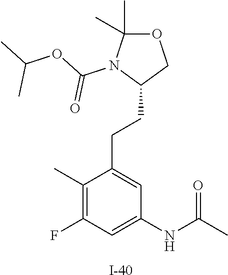 | 381.1 [M + 1]⁺ |
| Example 41 | 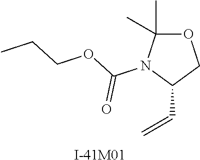 | 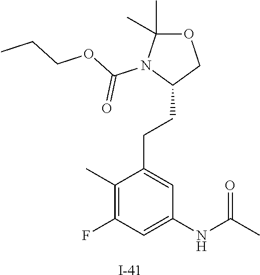 | 381.1 [M + 1]⁺ |
| Example 42 | 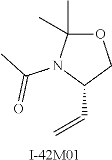 | 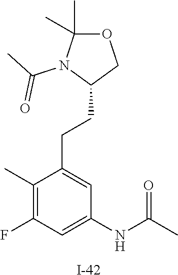 | 337.2 [M + 1]⁺ |
| Example 43 | 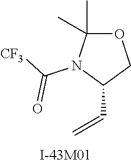 | 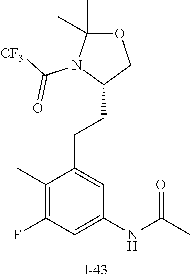 | 391.1 [M + 1]⁺ |
| Example 44 | 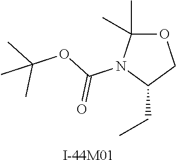 | 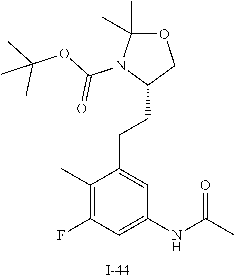 | 394.2 [M + 1]⁺ |
| Example 45 | 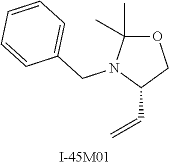 | 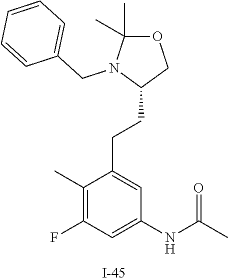 | 385.2 [M + 1]⁺ |

### Example 46: Preparation of Compound I-46

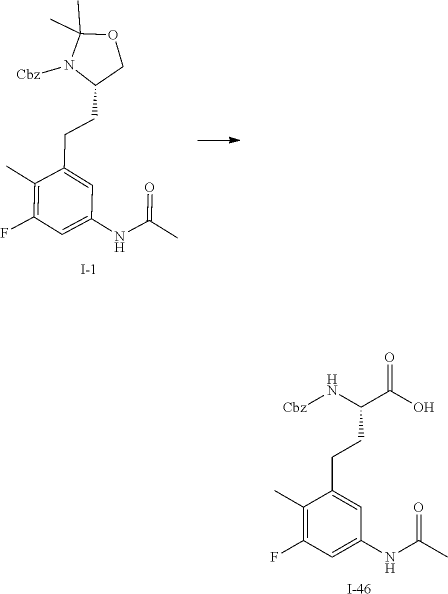

To a solution of I-1 (1.87 g, 4.4 mmol) in acetone (50 mL), add fresh Jones reagent (chromium trioxide, 3.2 g, 31.76 mmol dissolved in a small amount of water, add 4 mL of concentrated sulfuric acid and dilute with water to 12 mL) at 0° C. The reaction solution was warmed to room temperature and the reaction was continued to stir for 45 minutes. The above reaction solution was quenched with 6 mL of isopropyl alcohol and extracted with ethyl acetate. The organic phases were combined, washed with saturated ammonium chloride, washed with saturated sodium chloride solution, dried over anhydrous sodium sulfate, filtered, and concentrated under reduced pressure to obtain a crude product, which was purified through a silica gel column to obtain compound I-46 as a foamy solid (1.72 g, collected rate 98%). MS (ESI), m/z, 403.1 [M+1]⁺.

### Example 47: Preparation of Compound I-47

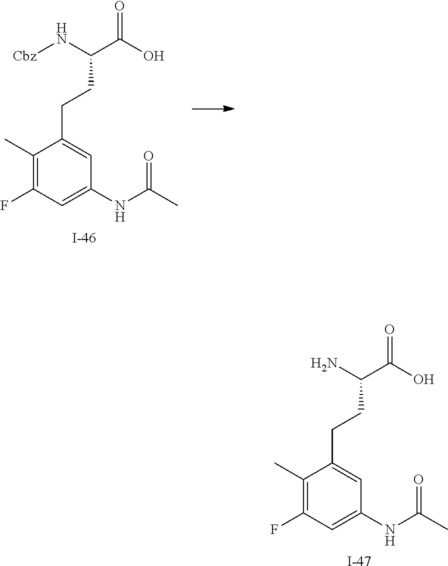

To a solution of I-46 (1.72 g, 4.3 mmol) in methanol (20 mL) was added 10% Pd/C (460 mg, 0.43 mmol) at room temperature. The reaction solution was backfilled with hydrogen and reacted under hydrogen atmosphere (hydrogen balloon) for 1 h. The mixture was filtered through celite, washed with a large amount of methanol, and concentrated to obtain compound I-47 as a gray solid (0.85 g, yield 74%).

MS (ESI), m/z, 269.1 [M+1]⁺.

### Example 48: Preparation of Compound I-48

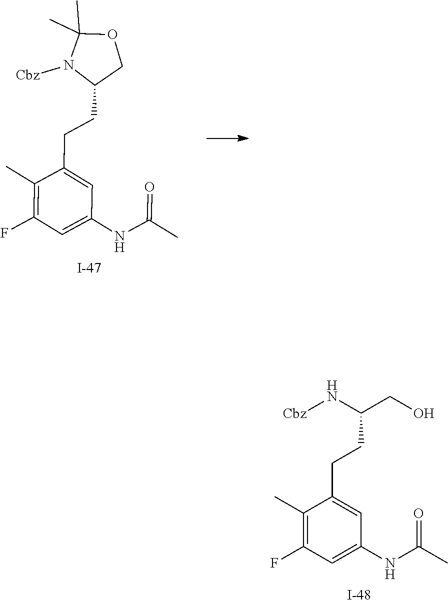

To a solution of I-47 (1.0 g, 2.34 mmol) in dichloromethane (10 mL) was added trifluoroacetic acid (2 mL) at 0° C. The reaction solution was warmed to room temperature and the reaction was continued to stir for 12 h. The above reaction solution was concentrated, diluted with water, neutralized with sodium bicarbonate solution, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-48 as an oily solid (0.9 g, yield 99%).

MS (ESI), m/z, 389.1 [M+1]⁺.

### Example 49: Preparation of Compound I-49

### Synthesis Method A

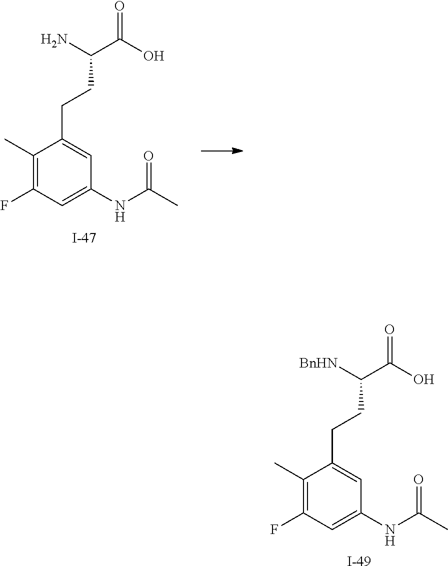

To a solution of I-3 (1.5 g, 5.60 mmol) in dioxane (20 mL) was added aqueous potassium carbonate solution (1.2 g dissolved in 20 mL water) and benzyl chloride (1.1 g) at room temperature and stirred the reaction 12 h. The reaction solution was filtered, and the filtrate was concentrated, adjusted to pH<7 with 2 N dilute hydrochloric acid, filtered, and dried under vacuum to obtain compound I-49 as a white solid (1.5 g, yield 75%).

MS (ESI), m/z, 359.1 [M+1]⁺.

### Synthesis Method B

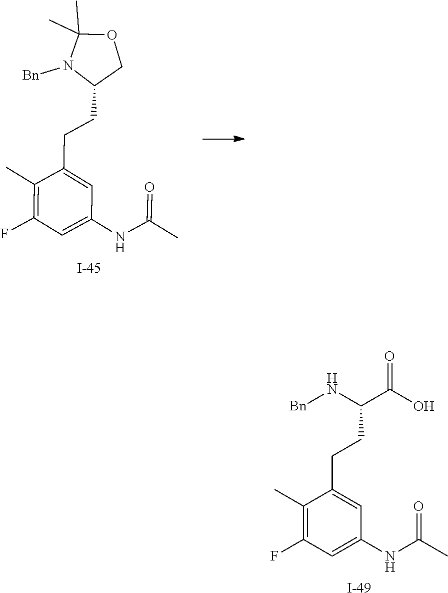

To a solution of I-45 (0.9 g, 2.3 mmol) in acetone (20 mL) was added fresh Jones reagent (chromium trioxide, 1.9 g, 18.8 mmol dissolved in a small amount of water, add 2 mL of concentrated sulfuric acid and dilute with water to 6 mL) at 0° C. The reaction solution was warmed to room temperature and the reaction was continued to stir for 1 h. The above reaction solution was quenched with 3 mL of isopropyl alcohol and extracted with ethyl acetate. The organic phases were combined, washed with saturated ammonium chloride, washed with saturated sodium chloride solution, dried over anhydrous sodium sulfate, filtered, and concentrated under reduced pressure to obtain a crude product, which was purified by silica gel column chromatography to obtain compound I-49 as a white solid (0.7 g, Yield 83%). MS (ESI), m/z, 359.1 [M+1]⁺.

### Examples 50-56D: Preparation of Compounds I-50 to I-56D

According to the synthetic method of Example 49, compounds I-50 to I-56D were prepared according to method A or B.

| Examples | raw materials | Structure | MS (ESI), m/z |
|---|---|---|---|
| Example 50 | Method A or B | 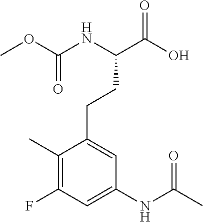 [SMILES: COC(=O)N[C@@H](CCc1cc(NC(C)=O)cc(F)c1C)C(=O)O] | 327.2 [M + 1]⁺ |
| | | I-50 | |
| Example 51 | Method A or B | 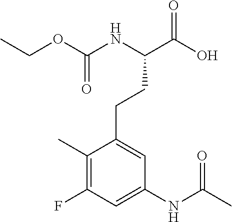 [SMILES: CCOC(=O)N[C@@H](CCc1cc(NC(C)=O)cc(F)c1C)C(=O)O] | 341.1 [M + 1]⁺ |
| | | I-51 | |
| Example 52 | Method A or B | 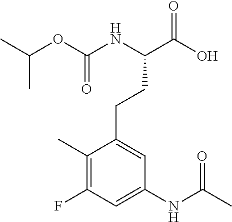 [SMILES: CC(=O)Nc1cc(F)c(C)c(CC[C@H](NC(=O)OC(C)C)C(=O)O)c1] | 355.2 [M + 1]⁺ |
| | | I-52 | |
| Example 53 | Method A or B | 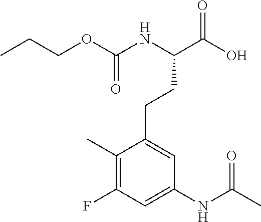 [SMILES: CCCOC(=O)N[C@@H](CCc1cc(NC(C)=O)cc(F)c1C)C(=O)O] | 355.2 [M + 1]⁺ |
| | | I-53 | |
| Example 54 | Method A or B | 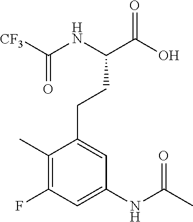 [SMILES: CC(=O)Nc1cc(F)c(C)c(CC[C@H](NC(=O)C(F)(F)F)C(=O)O)c1] | 365.2 [M + 1]⁺ |
| | | I-54 | |
| Example 55 | Method A or B | 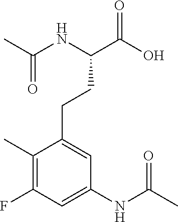 [SMILES: CC(=O)Nc1cc(F)c(C)c(CC[C@H](NC(C)=O)C(=O)O)c1] | 311.2 [M + 1]⁺ |
| | | I-55 | |
| Example 56 | Method B | 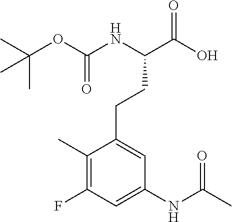 [SMILES: CC(=O)Nc1cc(F)c(C)c(CC[C@H](NC(=O)OC(C)(C)C)C(=O)O)c1] | 369.2 [M + 1]⁺ |
| | | I-56 | |
| Example 56A | Method A or B | 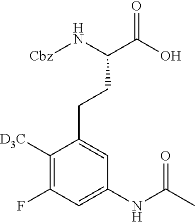 [SMILES: [2H]C([2H])([2H])c1c(F)cc(NC(C)=O)cc1CC[C@H](NC(=O)OCc1ccccc1)C(=O)O] | 406.3 [M + 1]⁺ |
| | | I-56A | |
| Example 56B | Method A or B | 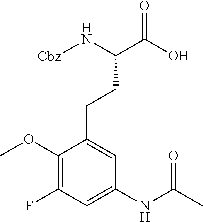 [SMILES: COc1c(F)cc(NC(C)=O)cc1CC[C@H](NC(=O)OCc1ccccc1)C(=O)O] | 419.2 [M + 1]⁺ |
| | | I-56B | |
| Example 56C | Method A or B | 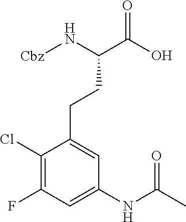 [SMILES: CC(=O)Nc1cc(F)c(Cl)c(CC[C@H](NC(=O)OCc2ccccc2)C(=O)O)c1] | 423.1 [M + 1]⁺ |
| | | I-56C | |
| Example 56D | Method A or B | 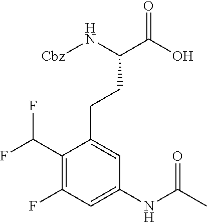 [SMILES: CC(=O)Nc1cc(F)c(C(F)F)c(CC[C@H](NC(=O)OCc2ccccc2)C(=O)O)c1] | 439.0 [M + 1]⁺ |
| | | I-56D | |

### Example 57: Preparation of Compound I-57

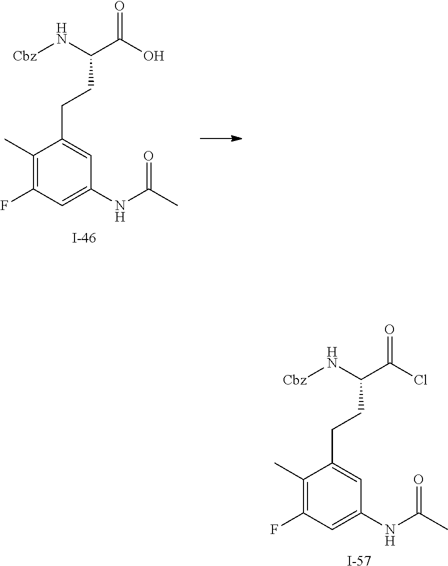

### Synthesis Method A

To a solution of I-46 (1.0 g, 2.5 mmol) in dichloromethane (20 mL) was added thionyl chloride (2.9 g, 24.8 mmol) dropwise at 0° C. under nitrogen protection. The above reaction solution was stirred for 0.5 h and directly concentrated to obtain crude product I-57, which was directly used in the next reaction.

Methanol quenched: MS (ESI), m/z, 417.1 [M+1]⁺.

### Synthesis Method B

To a solution of I-46 (1.0 g, 2.5 mmol) in dry dichloromethane (20 mL) was added dropwise oxalyl chloride (0.63 g, 5.0 mmol) and 2 drops of dry DMF at 0° C. under nitrogen atmosphere, and the reaction was stirred for 5-10 minutes, and the above reaction solution containing compound I-57 was used directly for the next reaction.

Methanol quenched: MS (ESI), m/z, 417.1 [M+1]⁺.

### Examples 58-66D: Preparation of Compounds I-58 to I-66D

According to the synthetic method of Example 57, compounds I-58 to I-66D were prepared according to their respective raw materials.

| Examples | raw materials | Structure | MS (ESI), m/z |
|---|---|---|---|
| Example 58 | Method A or B | 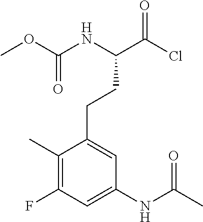 | Quenched with methanol: 341.2 [M + 1]⁺ |
| | | I-58 | |
| Example 59 | Method A or B | 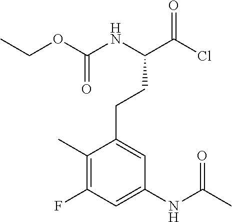 | Quenched with methanol: 355.1 [M + 1]⁺ |
| | | I-59 | |
| Example 60 | Method A or B | 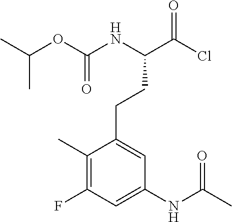 | Quenched with methanol: 369.2 [M + 1]⁺ |
| | | I-60 | |
| Example 61 | Method A or B | 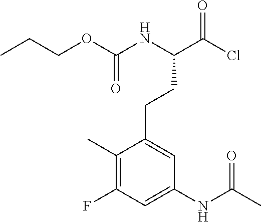 | Quenched with methanol: 369.2 [M + 1]⁺ |
| | | I-61 | |
| Example 62 | Method B | 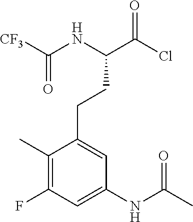 | Quenched with methanol: 379.2 [M + 1]⁺ |
| | | I-62 | |
| Example 63 | Method A or B | 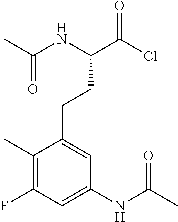 | Quenched with methanol: 325.2 [M + 1]⁺ |
| | | I-63 | |
| Example 64 | Method B | 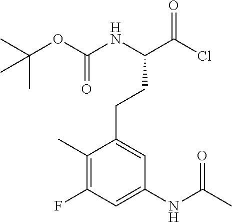 | Quenched with methanol: 383.2 [M + 1]⁺ |
| | | I-64 | |
| Example 65 | Method A or B | 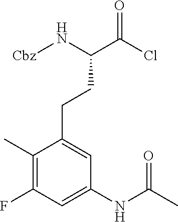 | Quenched with methanol: 417.2 [M + 1]⁺ |
| | | I-65 | |
| Example 66 | Method A or B | 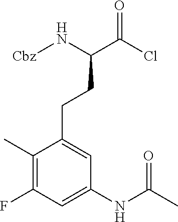 | Quenched with methanol: 417.2 [M + 1]⁺ |
| | | I-66 | |
| Example 66A | Method A or B | 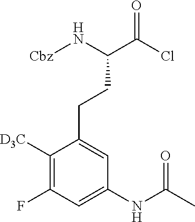 | Quenched with methanol: 420.1 [M + 1]⁺ |
| | | I-66A | |
| Example 66B | Method A or B | 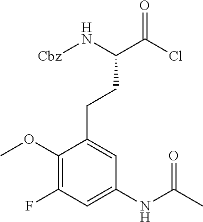 | Quenched with methanol: 433.3 [M + 1]⁺ |
| | | I-66B | |
| Example 66C | Method A or B | 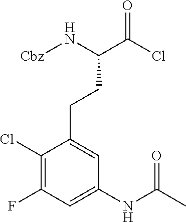 | Quenched with methanol: 437.1 [M + 1]⁺ |
| | | I-66C | |
| Example 66D | Method A or B | 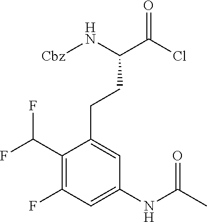 | Quenched with methanol: 453.2 [M + 1]⁺ |
| | | I-66D | |

### Example 67: Preparation of Compound I-67

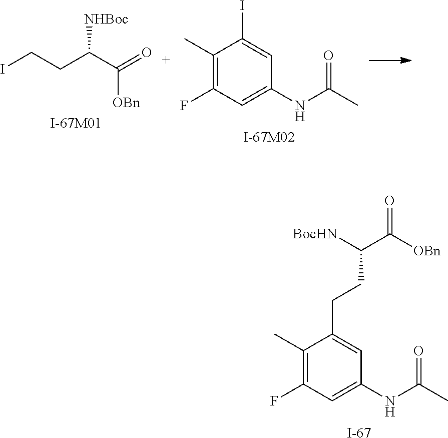

Zinc powder (200 mg, 3.4 mmol) was added to a dry nitrogen-protected one-neck flask, followed by dry DMF (10 mL), 1, 2-dibromoethane (33 mg, 0.18 mmol) and a catalytic amount of trimethylchlorosilane (4 mg). The mixture was stirred vigorously for 30 min at room temperature and allowed to stand. The supernatant was removed with a syringe and heated with a high-temperature gun under vacuum to continue activation for 1 min. The mixture was cooled to room temperature, I-67M01 (500 mg, 1.2 mmol) (Prepared according to the synthesis method of compound 9 in J. Org. Chem. 1998, 63, 7875-7884) was added dropwise, and stirred for 1 h. The above solution was transferred with syringe to a dry DMF solution (5 mL) of I-67M02 (174 mg, 0.6 mmol) (Prepared according to the synthesis method of Example 8 of patent TW201920078A), Pd₂(dba)₃ (27 mg, 0.03 mmol) and PEPPSI-IPr catalyst (41 mg, 0.06 mmol), and stirred at room temperature for 12 h. The reaction solution was filtered, the filtrate was diluted with water, and extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-67 (220 mg, yield 82%). MS (ESI), m/z, 459.2 [M+1]⁺.

### Example 68: Preparation of Compound I-68

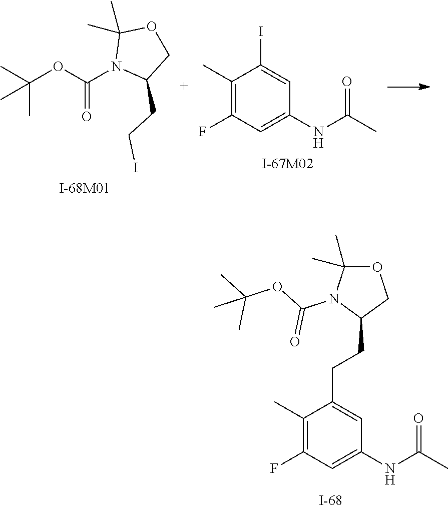

### Synthesis Method A

To a flask was added dry THF (20 mL), pretreated magnesium turnings (20 mg, 0.85 mmol, washed with 10% hydrochloric acid for 30 minutes, quickly filtered, rinsed with acetone, dried in vacuum, and used directly) and iodine (7 mg, 0.003 mmol) under nitrogen atmosphere. Then a THF solution (10 mL) of I-68M01 (242 mg, 0.68 mmol) was added dropwise, and the temperature was controlled at 20-30° C. After addition was completed, the mixture was stirred for 1 h and allowed to stand. The supernatant was transferred to a dry THE solution (10 mL) containing I-67M02 (100 mg, 0.34 mmol), Pd₂(dba)₃ (31 mg, 0.03 mmol) and S-Phos catalyst (28 mg, 0.06 mmol), and heated to 50° C. for 12 h. The reaction solution was quenched by ammonium chloride aqueous solution, filtered, and the filtrate was extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-68 (80 mg, yield 60%).

MS (ESI), m/z, 395.2 [M+1]⁺.

### Synthesis Method B

To a flask was added dry THF (20 mL), pretreated magnesium turnings (20 mg, 0.85 mmol, washed with 10% hydrochloric acid for 30 minutes, quickly filtered, rinsed with acetone, dried in vacuum, and used directly) and iodine (7 mg, 0.003 mmol) under nitrogen atmosphere. To the above mixture was added dropwise solution of I-68M01 (242 mg, 0.68 mmol) in THE solution (10 mL) was added and the temperature was controlled at 20-30° C. After the addition was completed, the mixture was stirred for 1 h and allowed to stand. The supernatant was transferred to another reaction flask. Zinc chloride (0.5 M in THF, 0.7 mL, 0.51 mmol) solution was added dropwise under nitrogen atmosphere, stirred for 0.5 h, and then added I-67M02 (100 mg, 0.34 mmol) and Pd₂(dba)₃ (31 mg, 0.03 mmol). and PEPPSI-IPr catalyst (46 mg, 0.07 mmol), and stirred for 12 h at room temperature. The reaction solution was quenched with water, filtered, and the filtrate was extracted with ethyl acetate. The organic phases were combined, washed with saturated sodium chloride solution, dried over anhydrous sodium sulfate, filtered, and concentrated under reduced pressure to obtain a crude product, which was purified by silica gel column chromatography to obtain compound I-68 (95 mg, yield 70%).

MS (ESI), m/z, 395.2 [M+1]⁺.

### Example 69: Preparation of Compound I-69

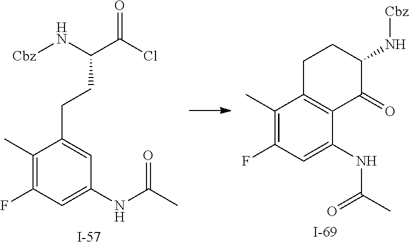

To a solution I-57 (500 mg, 1.2 mmol) in dry dichloromethane was added anhydrous aluminum trichloride (540 mg, 3.6 mmol) in portions under nitrogen atmosphere in an ice-water bath. The mixture was slowly warmed to room temperature and stirred for 1 h, poured into ice water, and extracted with dichloromethane. The organic phases were combined, washed with saturated sodium chloride solution, dried over anhydrous sodium sulfate, filtered, and concentrated under reduced pressure to obtain a crude product, which was purified by silica gel column chromatography to obtain compound I-69 (400 mg, yield 87%).

MS (ESI), m/z, 385.2 [M+1]⁺.

### Example 70: Preparation of Compound I-70

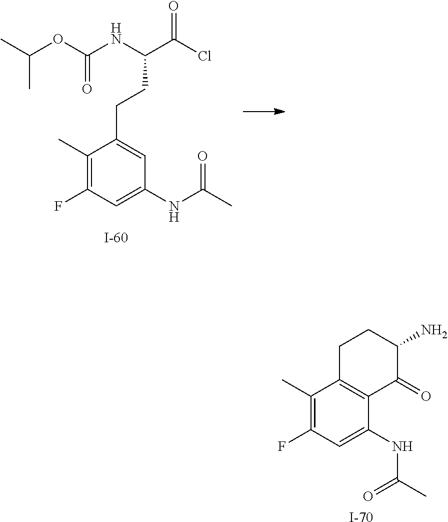

To the dry dichloromethane solution of I-60 (500 mg, 1.3 mmol) was added anhydrous aluminum trichloride (750 mg, 5.0 mmol) in portions in an ice-water bath under nitrogen atmosphere. The mixture was slowly warmed to room temperature and stirred for 1 h, poured into ice water, and adjusted to alkaline with 2 N sodium hydroxide solution. The mixture was extracted with dichloromethane, combined the organic phases, washed with saturated sodium chloride solution, dried with anhydrous sodium sulfate, filtered, and concentrated under reduced pressure to obtain a crude product, which was purified by silica gel column chromatography to obtain compound I-70 (280 mg, yield 83%).

MS (ESI), m/z, 251.2 [M+1]⁺.

### Example 71: Preparation of Compound I-71

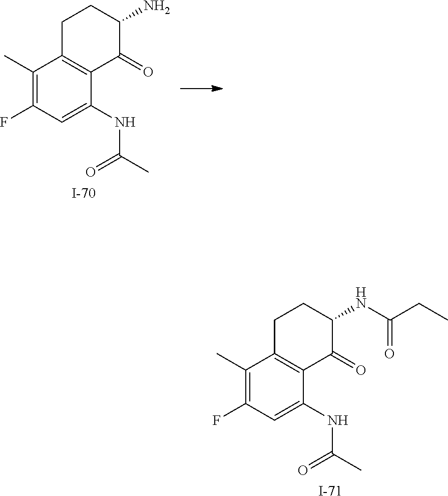

To a solution of I-70 (100 mg, 0.4 mmol) in dry dichloromethane were added propionic anhydride (104 mg, 0.8 mmol) and triethylamine (80 mg, 0.8 mmol) in an ice-water bath under nitrogen atmosphere. The reaction solution was stirred for 0.5 h, quenched with water, and extracted with dichloromethane. The organic phases were combined, washed with saturated sodium chloride solution, dried over anhydrous sodium sulfate, filtered, and concentrated under reduced pressure to obtain compound I-71 (110 mg, yield 94%).

MS (ESI), m/z, 307.2 [M+1]⁺.

### Example 72: Preparation of Compound I-72

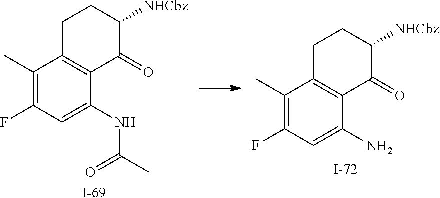

To a methanol solution (5 mL) of I-69 (100 mg, 0.26 mmol) was added 3 N dilute hydrochloric acid (3 mL) in an ice-water bath under a nitrogen atmosphere. The mixture was stirred at room temperature for 12 h, cooled to 0° C., basified with 2 N sodium hydroxide, and extracted with dichloromethane. The organic phases were combined, washed with saturated sodium chloride solution, dried over anhydrous sodium sulfate, filtered, and concentrated under reduced pressure to obtain a crude product, which was purified by silica gel column chromatography to obtain compound I-72 (80 mg, yield 90%).

MS (ESI), m/z, 343.2 [M+1]⁺.

### Examples 73-84D: Preparation of Compounds I-73 to I-84D

According to the synthesis method of Examples 70-72, compounds I-73 to I-84D were prepared according to their respective raw materials:

| Examples | Structure | MS (ESI), m/z |
|---|---|---|
| Example 73 | 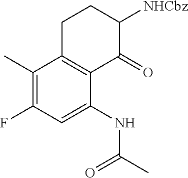 [SMILES: CC(=O)Nc1cc(F)c(C)c2c1C(=O)C(NC(=O)OCc1ccccc1)CC2] | 385.2 [M + 1]⁺ |
| | I-73 | |
| Example 74 | 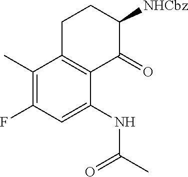 [SMILES: CC(=O)Nc1cc(F)c(C)c2c1C(=O)[C@H](NC(=O)OCc1ccccc1)CC2] | 385.2 [M + 1]⁺ |
| | I-74 | |
| Example 75 | 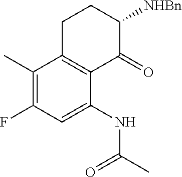 | 341.1 [M + 1]⁺ |
| | I-75 | |
| Example 76 | 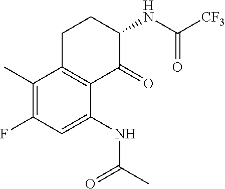 [SMILES: CC(=O)Nc1cc(F)c(C)c2c1C(=O)[C@@H](NC(=O)C(F)(F)F)CC2] | 347.2 [M + 1]⁺ |
| | I-76 | |
| Example 77 | 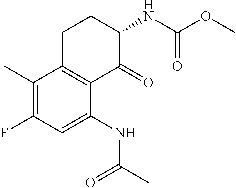 [SMILES: COC(=O)N[C@H]1CCc2c(C)c(F)cc(NC(C)=O)c2C1=O] | 309.1 [M + 1]⁺ |
| | I-77 | |
| Example 78 | 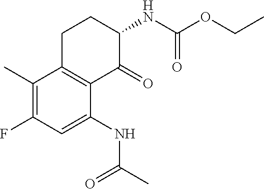 [SMILES: CCOC(=O)N[C@H]1CCc2c(C)c(F)cc(NC(C)=O)c2C1=O] | 323.2 [M + 1]⁺ |
| | I-78 | |
| Example 79 | 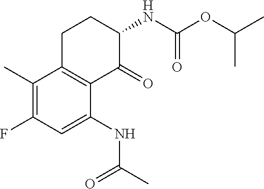 [SMILES: CC(=O)Nc1cc(F)c(C)c2c1C(=O)[C@@H](NC(=O)OC(C)C)CC2] | 337.1 [M + 1]⁺ |
| | I-79 | |
| Example 80 | 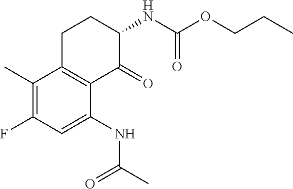 [SMILES: CCCOC(=O)N[C@H]1CCc2c(C)c(F)cc(NC(C)=O)c2C1=O] | 337.1 [M + 1]⁺ |
| | I-80 | |
| Example 81 | 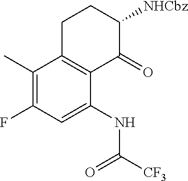 [SMILES: Cc1c(F)cc(NC(=O)C(F)(F)F)c2c1CC[C@H](NC(=O)OCc1ccccc1)C2=O] | 439.2 [M + 1]⁺ |
| | I-81 | |
| Example 82 | 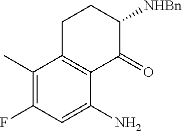 | 299.1 [M + 1]⁺ |
| | I-82 | |
| Example 83 | 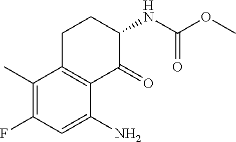 | 267.1 [M + 1]⁺ |
| | I-83 | |
| Example 84 | 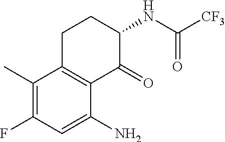 [SMILES: Cc1c(F)cc(N)c2c1CC[C@H](NC(=O)C(F)(F)F)C2=O] | 305.1 [M + 1]⁺ |
| | I-84 | |
| Example 84A | 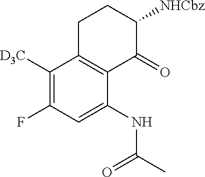 [SMILES: [2H]C([2H])([2H])c1c(F)cc(NC(C)=O)c2c1CC[C@H](NC(=O)OCc1ccccc1)C2=O] | 388.2 [M + 1]⁺ |
| | I-84A | |
| Example 84B | 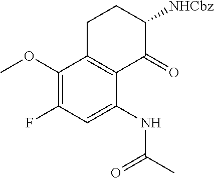 [SMILES: COc1c(F)cc(NC(C)=O)c2c1CC[C@H](NC(=O)OCc1ccccc1)C2=O] | 401.1 [M + 1]⁺ |
| | I-84B | |
| Example 84C | 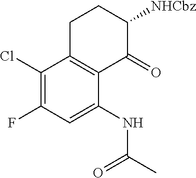 [SMILES: CC(=O)Nc1cc(F)c(Cl)c2c1C(=O)[C@@H](NC(=O)OCc1ccccc1)CC2] | 405.3 [M + 1]⁺ |
| | I-84C | |
| Example 84D | 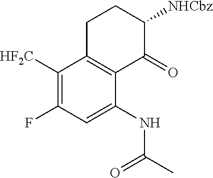 | 420.1 [M + 1]⁺ |
| | I-84D | |

### Example 85: Preparation or Compound I-85

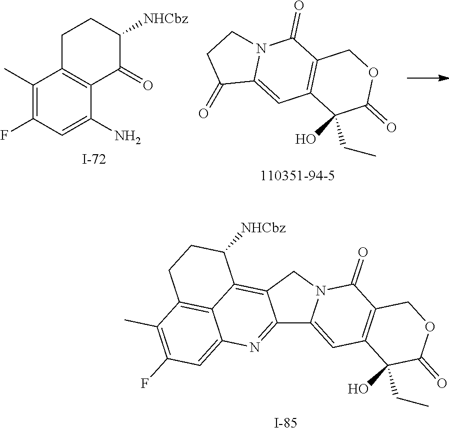

To a solution of I-72 (300 mg, 0.88 mmol) in toluene (8 mL) was added the compound of CAS number 110351-94-5 (254 mg, 0.96 mmol) and p-toluenesulfonic acid monohydrate (50 mg, 0.26 mmol) or pyridine 4-methylbenzenesulfonate (66 mg, 0.26 mmol), the reaction solution was heated to 110° C. and stirred for 12 h. After the reaction was completed, it was concentrated and directly purified by column chromatography to obtain a crude product, which was then slurried with ethanol and purified to obtain compound I-85 (410 mg, yield 82%). MS (ESI), m/z, 570.1 [M+H]⁺.

### Example 86: Preparation of Compound I-86

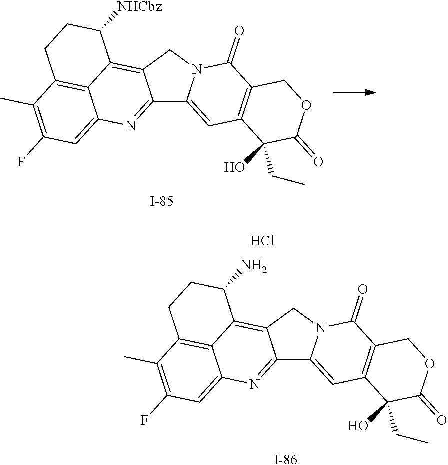

To a solution of I-85 (200 mg, 0.35 mmol) in methanol (5 mL) and 6 N dilute hydrochloric acid (10 mL) was added 10% Pd/C (20 mg) at room temperature. The mixture was backfilled with hydrogen and reacted in a hydrogen atmosphere (hydrogen balloon) for 6 h. The mixture was filtered with celite, washed with methanol and dilute hydrochloric acid aqueous solution, concentrated, and slurried with ethanol to obtain compound I-86 (150 mg, yield 91%).

MS (ESI), m/z, 436.3 [M+1]⁺.

¹H NMR (400 MHz, DMSO) δ 8.49 (s, 3H), 7.88 (d, J =10.8 Hz, 1H), 7.34 (s, 1H), 6.55 (s, 1H), 5.88-5.71 (m, 1H), 5.43 (d, J=21.2 Hz, 3H), 5.06 (s, 1H), 3.44 (dt, J=12.0, 7.0 Hz, 1H), 3.14 (t, J=13.5 Hz, 1H), 2.55 (s, 1H), 2.42 (s, 3H), 2.17 (dd, J=18.1, 9.0 Hz, 1H), 1.95-1.80 (m, 2H), 0.88 (t, J=7.3 Hz, 3H).

1. A compound represented by formula (I), its stereoisomer or its pharmaceutically acceptable salt: 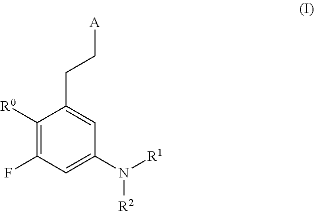
wherein,
A is selected from 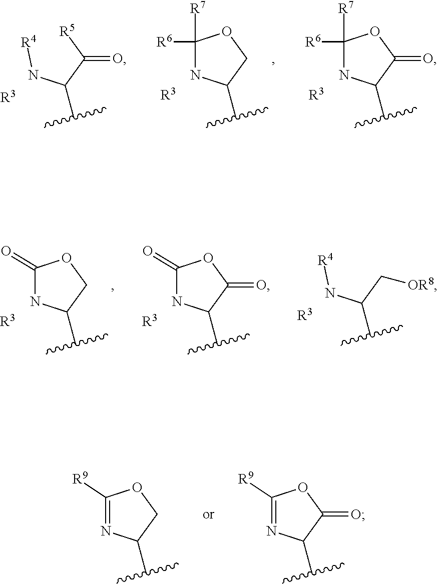
the chiral configuration in structure A can be R configuration, S configuration, or a mixture of R configuration and S configuration;
R⁰ is selected from hydroxyl, alkoxy, sulfhydryl, alkylthio, halogen, nitro, substituted or unsubstituted amino, C1-C6 alkyl, C1-C6 cycloalkyl, C1-C6 haloalkyl, C1-C6 deuterium alkyl, C2-C6 alkenyl or C2-C6 alkynyl;
R¹ and R² are each independently selected from hydrogen or amino protecting groups;
R³ and R⁴ are each independently selected from hydrogen or amino protecting groups;
R⁵ is selected from hydrogen, hydroxyl, alkoxy, alkylthio, halogen or substituted or unsubstituted amino;
R⁶ and R⁷ are each independently selected from hydrogen, alkyl, haloalkyl or deuterated alkyl;
alternatively, R⁶ and R⁷ together with the carbon atom linked thereto form a 3-7 membered cycloalkyl or heterocycloalkyl group;
R⁸ is selected from hydrogen or hydroxyl protecting group;
R⁹ is selected from hydrogen, alkoxy, alkylthio, C1-C6 alkyl, C1-C6 haloalkyl, aryl or heteroaryl.

2. The compound shown in formula (I) according to claim 1, its stereoisomer or its pharmaceutically acceptable salt, characterized in that the compound shown in formula (I) is selected from the following structures without limitation:

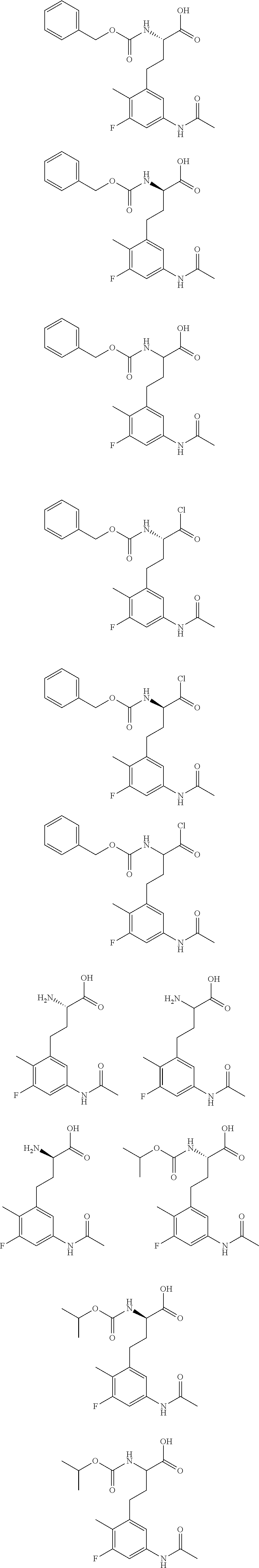

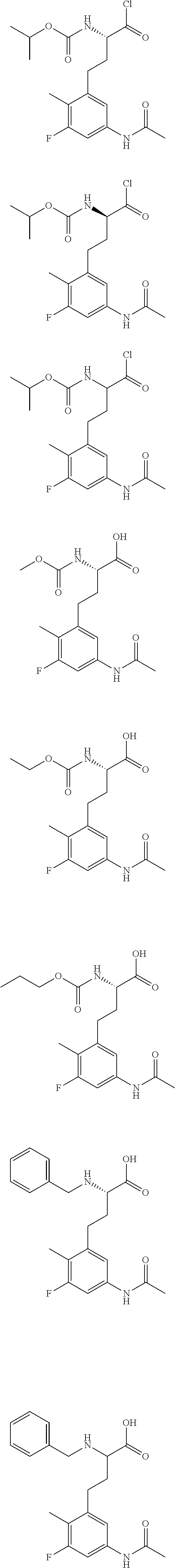

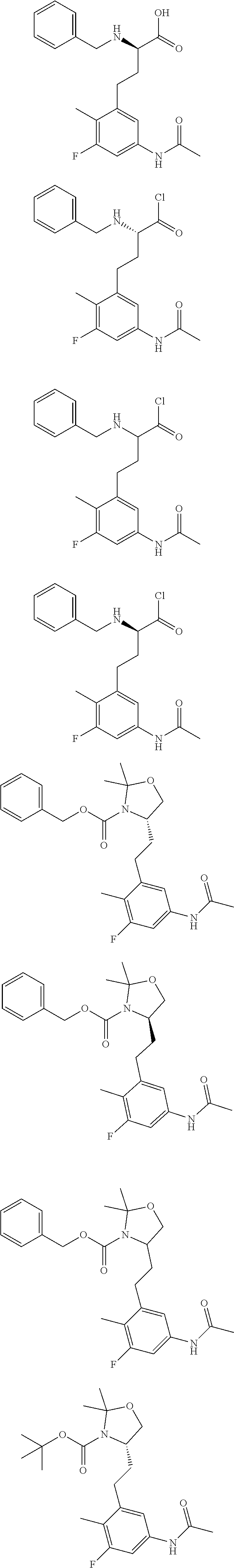

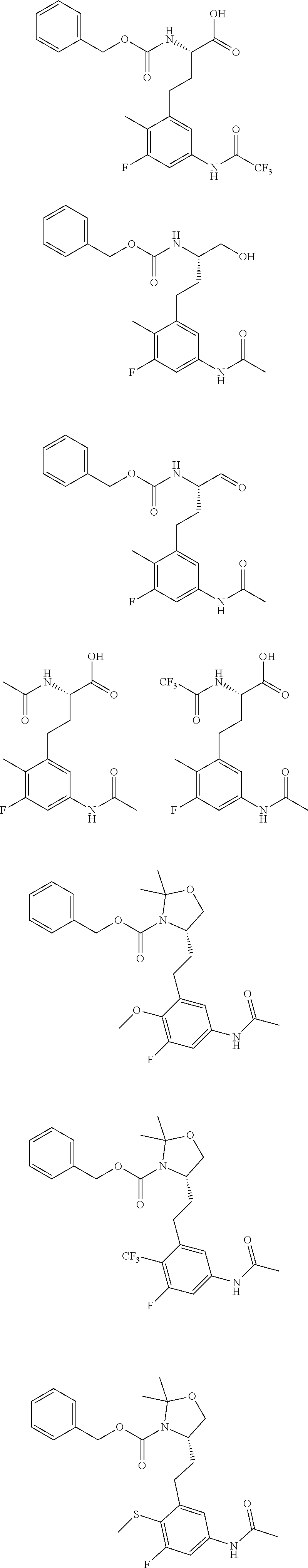

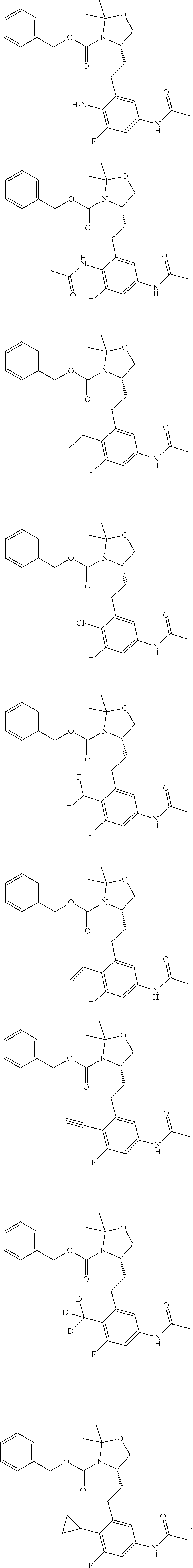

3. A method for preparing a compound shown in formula (I) according to claim 1, which comprises the following steps: compound 2 is first reacted with a borane reagent to form a boron intermediate, and then coupled with compound 1 with metal catalyst and base, the reaction formula is as follows: 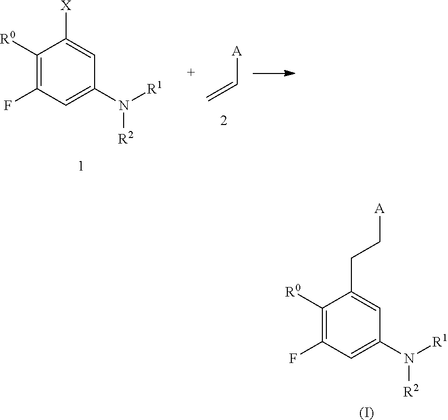
wherein, X is selected from Cl, Br or I.

4. The preparation method of the compound shown in formula (I) according to claim 3 is characterized in that: the borane reagent is 9-borabicyclo[3.3.1]nonane (9-BBN), dicyclohexylborane (Cy₂BH), catecholborane (CatBH), pinacol borane (PinBH) and disiamylborane (Sia₂BH), borane tetrahydrofuran complex, borane dimethyl sulfide complex or diisopinenylborane (Ipc₂BH).

5. The preparation method of the compound shown in formula (I) according to claim 3, characterized in that: the coupling reaction is a Suzuki reaction, and the metal catalyst is selected from Pd(PPh₃)₄, PdCl₂(dppf), PdCl₂(dppf)CH₂Cl₂, PdCl₂(PPh₃)₂, Pd₂(dba)₃/XPhos, Pd₂(dba)₃/SPhos, Pd₂(dba)₃/XantPhos, Pd(OAc)₂/PCy₃, PdCl₂(dppe) or PdCl₂(dppp)₄; the base is selected from one or more of potassium phosphate, sodium carbonate, cesium carbonate, potassium carbonate, sodium bicarbonate, potassium fluoride, cesium fluoride, sodium hydroxide, potassium hydroxide, sodium tert-butoxide, potassium tert-butoxide, potassium acetate, sodium acetate, triethylamine, diisopropylethylamine, and DBU; the solvent is selected from one or more of tetrahydrofuran, methyltetrahydrofuran, diethyl ether, toluene, DMF, DMA, DME, NMP, DMSO, dioxane, 1,2-dichloroethane, tert-butanol, water, and acetonitrile.

6. A preparation method of the compound shown in formula (I) according to claim 1, characterized in that it comprises the following steps: compound 3 is first mixed with zinc to form a zinc intermediate with catalyst, and then compound 1 is coupled, and the reaction formula is as follows: 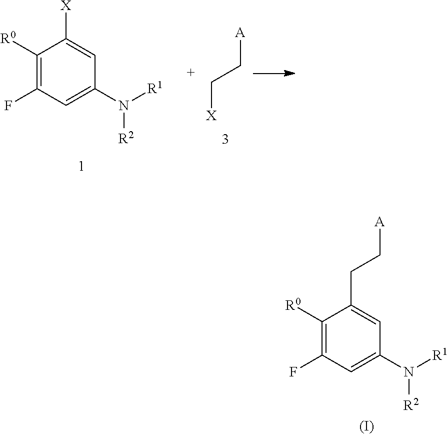
wherein, X is selected from Cl, Br or I.

7. The preparation method of the compound shown in formula (I) according to claim 6, characterized in that: the catalyst used to form the zinc intermediate is selected from one or more of 1,2-dibromoethane, TMSCl, iodine, lithium chloride or lithium bromide; the coupling reaction is a Negishi coupling reaction, and the metal catalyst is selected from Pd(PPh₃)₄, PdCl₂(dppf), PdCl₂(dppf)CH₂Cl₂, PdCl₂(PPh₃)₂, Pd₂(dba)₃/XPhos, Pd₂(dba)₃/SPhos, Pd₂(dba)₃/XantPhos, Pd(OAc)₂/PCy₃, Pd₂(dba)₃/P(o-tol)₃, Pd₂(dba)₃/P(o-tol)₃, Pd₂(dba)₃/P(2-furyl)₃, Pd₂(dba)₃/SPhos, Pd₂(dba)₃/RuPhos, PEPPSI-IPr, PEPPSI-IPent, NiCl₂(PPh₃)₂; the solvent is selected from one or more of tetrahydrofuran, methyltetrahydrofuran, toluene, DMF, DMA, DME, NMP, DMSO, dixoane, 1,2-dichloroethane, tert-butanol, water, and acetonitrile.

8. A method for preparing a compound as shown in formula (I) according to claim 1, characterized in that it comprises the following steps: compound 3 is first reacted with magnesium to form a magnesium intermediate, or is exchanged with a Grignard reagent to form magnesium intermediate, and then coupled with compound 1, the reaction formula is as follows: 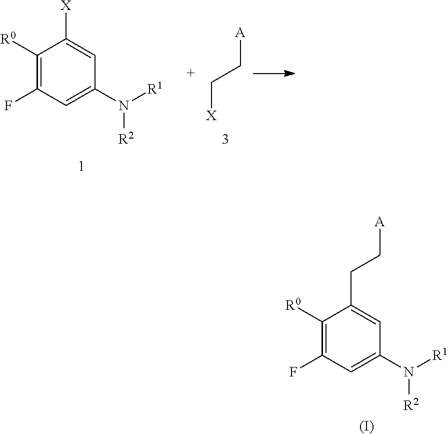
wherein, X is selected from Cl, Br or I.

9. The preparation method of the compound shown in formula (I) according to claim 8, characterized in that: the Grignard reagent used to form the magnesium intermediate is selected from isopropyl magnesium chloride, isopropyl magnesium bromide, isopropyl magnesium bromide, and isopropyl magnesium chloride lithium chloride complex, ethyl magnesium bromide, ethyl magnesium chloride, phenyl magnesium chloride, phenyl magnesium bromide; the coupling reaction is Kumada coupling reaction, and the metal catalyst is selected from Pd(PPh₃)₄, NiCl₂(dppp), NiCl₂(dppe), NiCl₂(PPh₃)₂, ferric acetylacetonate, nickel(II) acetylacetonate, FeCl₃, CoCl₂, Pd(PPh₃)₄, PdCl₂(dppf), PdCl₂(dppf) CH₂Cl₂, Pd(OAc)₂/PCy₃, Pd₂(dba)₃/SPhos; the solvent is selected from tetrahydrofuran, methyltetrahydrofuran, diethyl ether, toluene, DMF, DMA, DME, NMP, DMSO, dioxane, 1,2-dichloroethane, tert-butanol, water, and acetonitrile.

10. A method for preparing a compound shown in formula (I) according to claim 1, characterized in that it comprises the following steps: compound 3 is first reacted with magnesium to form a magnesium intermediate, or is exchanged with a Grignard reagent to form magnesium intermediate, and then exchanged with the zinc reagent to form a zinc intermediate, which is then coupled with compound 1, the reaction formula is as follows: 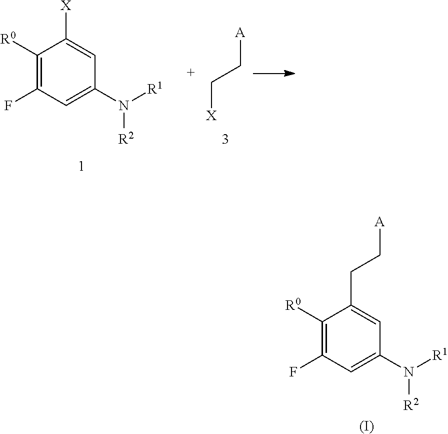
wherein, X is selected from Cl, Br or I.

11. The preparation method of the compound shown in formula (I) according to claim 10, characterized in that: the zinc reagent is selected from zinc chloride, zinc chloride-TMEDA, zinc bromide, zinc iodide, zinc methoxide; the coupling reaction is Negishi coupling reaction, and the metal catalyst is selected from Pd(PPh₃)₄, PdCl₂(dppf), PdCl₂(dppf)CH₂Cl₂, PdCl₂(PPh₃)₂, Pd₂(dba)₃/XPhos, Pd₂(dba)₃/SPhos, Pd₂(dba)₃/XantPhos, Pd(OAc)₂/PCy₃, Pd₂(dba)₃/P(o-tol)₃, Pd₂(dba)₃/P(o-tol)₃, Pd₂(dba)₃/P(2-furyl)₃, Pd₂(dba)₃/SPhos, Pd₂(dba)₃/RuPhos, PEPPSI-IPr, PEPPSI-IPent, NiCl₂(PPh₃)₂;
or, R⁰ is methyl.

12. (canceled)

13. A method for preparing a compound of formula (II), a stereoisomer thereof or a pharmaceutically acceptable salt thereof from a compound of formula (I), characterized by comprising the following steps: converting the compound of formula (I) into a compound of formula (II), and the reaction formula is as follows: 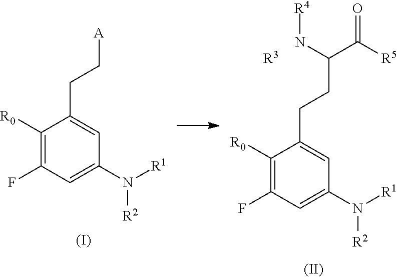
wherein R⁵ is selected from hydroxyl or halogen;
R⁰, R¹, R², R³, R⁴ and A are defined as in claim 1.

14. The method for preparing the compound represented by formula (II) according to claim 13, wherein R⁰ is methyl.

15. A method for preparing a compound of formula (III), a stereoisomer thereof or a pharmaceutically acceptable salt thereof from a compound of formula (II), characterized in that the compound of formula (II) is intramolecular cyclized with catalyst to generate a compound of formula (III), and the reaction formula is as follows: 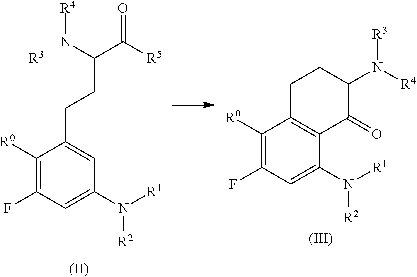
wherein R⁵ is selected from hydroxyl or halogen;
R⁰, R¹, R², R³ and R⁴ are each defined as in claim 1;
the chiral configuration of the compound (III) can be R configuration, S configuration or a mixture of R configuration and S configuration.

16. The preparation method of the compound represented by formula (III) according to claim 15, characterized in that: the catalyst used for ring closing is selected from one or more of anhydrous aluminum trichloride, tin tetrachloride, and titanium tetrachloride, ferric chloride, boron trifluoride ether, trifluoroacetic anhydride, concentrated sulfuric acid, and hexafluoroisopropanol;
or, R⁰ is methyl.

17. (canceled)

18. A method for preparing a compound of formula (IV), its stereoisomer or a pharmaceutically acceptable salt thereof from a compound represented by formula (III), characterized in that it includes the following steps:
a. selectively deprotect the compound represented by formula (III) to obtain compound 4;
b. compound 4 and compound 5 are cyclized with catalyst to form compound 6;
c. removing the protective group from the compound 6 to obtain the compound of formula (IV), its stereoisomer or its pharmaceutically acceptable salt; 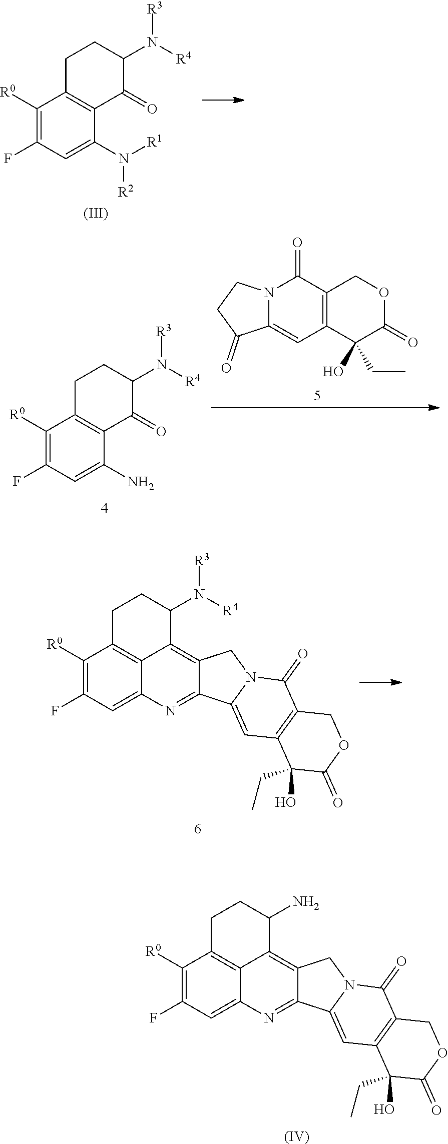
wherein,
R⁰, R¹, R², R³ and R⁴ are each as defined in claim 1.

19. The method for preparing the compound represented by formula (IV) according to claim 18, wherein R⁰ is methyl;
or, the chiral configuration of compound (III) is R configuration or S configuration; the chiral configuration of compound 4 is R configuration or S configuration.

20. (canceled)

21. A camptothecin derivative intermediate represented by formula (IIIa-IIIc), a stereoisomer thereof or a pharmaceutically acceptable salt thereof: 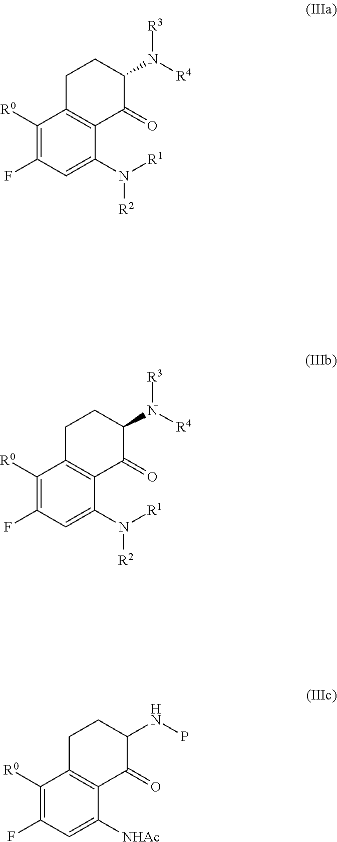
wherein, R⁰, R¹, R², R³ and R⁴ are each defined as described in claim 1, and P is an amino protecting group other than acetyl.

22. The camptothecin derivative intermediate represented by the formula (IIIa-IIIb), its stereoisomer or its pharmaceutically acceptable salt according to claim 21, wherein the camptothecin derivative intermediate represented by the formula (IIIa-IIIb) is selected from the following structures:

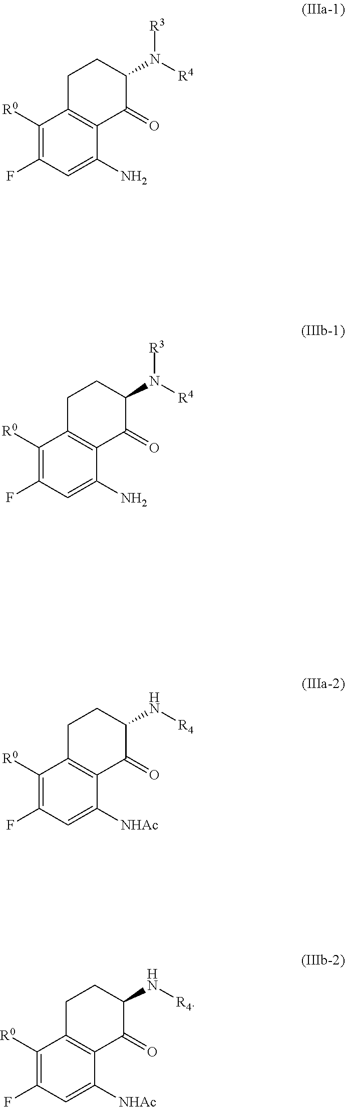

23. (canceled)

24. The camptothecin derivative intermediate represented by formula (IIIa-IIIc) according to claim 21, its stereoisomer or its pharmaceutically acceptable salt, wherein R⁰ is methyl.
